SEQUENCE LISTING (4 sequences)

SEQ ID NO: 1 - DNA, 21 bp, Artificial Sequence
  Description of Artificial Sequence Synthetic oligonucleotide
cagacgtgtgctcttccgatc

SEQ ID NO: 2 - DNA, 26 bp, Artificial Sequence
  Description of Artificial Sequence Synthetic oligonucleotide
ggctcggagatgtgtataagagacag

SEQ ID NO: 3 - DNA, 22 bp, Artificial Sequence
  Description of Artificial Sequence Synthetic oligonucleotide
cagacgtgtgctcttccgatct

SEQ ID NO: 4 - DNA, 25 bp, Artificial Sequence
  Description of Artificial Sequence Synthetic oligonucleotide
aaaaaaaaaaaaaaaaaaaaaaaaa

(19) United States
(12) Patent Application Publication
(10) Pub. No.: US 2024/0125797 A1
(43) Pub. Date: Apr. 18, 2024

(54) QUANTIFICATION OF CELLULAR PROTEINS USING BARCODED BINDING MOIETIES

(71) Applicants: CZ Biohub SF, LL, San Francisco, CA (US); The Board of Trustees of the Leland Stanford Junior University, Stanford, CA (US)

(72) Inventors: William J. Greenleaf, Stanford, CA (US); Amy F. Chen, Stanford, CA (US)

(21) Appl. No.: 18/262,519

(22) PCT Filed: Jan. 26, 2022
(86) PCT No.: PCT/US2022/013885

§ 371 Date: Jul. 21, 2023

Related U.S. Application Data
(60) Provisional application No. 63/141,818, filed on Jan. 26, 2021.

(51) Int. Cl. G01N 33/68

(52) U.S. Cl. CPC G01N 33/6854

(57) ABSTRACT

The invention provides compositions and methods for quantifying cellular molecules of interest, e.g., intracellular proteins, using oligonucleotide-target binding moiety conjugates.

### CROSS-REFERENCE TO RELATED APPLICATIONS

This application claims priority to U.S. Provisional Application No. 63/141,818, filed Jan. 26, 2021, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under contracts GM135996, HG007735, and HG009436 awarded by the National Institutes of Health. The Government has certain rights in the invention.

### BACKGROUND

The development of technologies to perform multiple genomic assays on a single cell is a relatively new area of research that has progressed rapidly in the past few years. These techniques have a range of applications, including the ability to obtain multiple genomic data sets (e.g., transcriptome and epigenome) from small amounts of primary tissue, profile cellular heterogeneity within a sample, and answer fundamental questions about gene regulation by examining the relationship between epigenetic and transcriptional state in cells.

Although assays for measuring RNA transcripts, chromatin accessibility, and cell surface protein abundance in single cells have been published and commercialized, a method for measuring cytoplasmic and nuclear protein levels has not yet been developed. The present invention provides such methods and compositions.

### BRIEF SUMMARY

Described here are compositions and methods to quantify cellular targets, e.g., proteins produced by a single cell, using a population of conjugate molecules in which target binding moieties are conjugated to oligonucleotides that comprise a barcode sequence, wherein the barcode sequence is specific for the target binding moiety, i.e., differs in sequence from the barcode sequence of conjugates that have different target binding moieties. The oligonucleotide components of the conjugates are coated with a nucleic acid binding protein, e.g., a sequence non-specific nucleic acid binding protein, or two or more nucleic acid binding proteins, that block the non-specific binding interactions of the oligonucleotide with cellular molecules and facilitate entry of the oligonucleotides into the cell and/or nucleus. Use of such conjugates provides highly sensitive and specific quantification of cellular proteins, including cytoplasmic and nuclear proteins.

In one aspect, the disclosure provides a method of quantifying the levels of a plurality of cellular proteins present in a cell, the method comprising
incubating a cell, e.g., a permeabilized cell with a population of binding moiety-oligonucleotide conjugates comprising a plurality of conjugates in which each conjugate comprises a target binding moiety that specifically binds to a cellular protein to be quantified conjugated to an oligonucleotide that comprises a target-binding-moiety barcode sequence, said target-binding-moiety barcode sequence differing in sequence from target-binding-moiety barcode sequences contained in oligonucleotide components of conjugates that comprise different target binding moieties, wherein the plurality of conjugates comprise a nucleic acid binding protein, e.g., a sequence non-specific nucleic acid binding protein, such as Escherichia coli SSB or T4 gene 32 protein, coated onto each oligonucleotide component of the conjugates; and
quantifying the level of barcode sequences for each binding moiety associated with the single cell, thereby quantifying the level of each of the cellular proteins bound to the target binding moiety. In some embodiments, the method further comprising performing an amplification reaction to amplify oligonucleotide sequences of conjugates bound to cellular proteins to obtain an amplification product. In some embodiments, the step of quantifying comprises a quantification amplification reaction, such as quantitative PCR. In some embodiments, the quantifying step comprises massively parallel sequencing. In some embodiments, the oligonucleotide comprises a detectable label, such as a fluorescent label; or the oligonucleotide hybridizes to a complementary oligonucleotide that comprises a detectable label, such as a fluorescent label. In some embodiments, the method further comprises detecting a signal from the label. In some embodiments, the method comprises detecting a signal from a fluorescent label to localize the position of the target binding moiety in the cell. In some embodiments, the method comprises quantifying the signal from the label, e.g., fluorescent label. In some embodiments, the binding moiety comprises an antibody, or a binding fragment thereof, that specifically binds to the target cellular protein. In some embodiments, the binding moiety comprises an aptamer that specifically binds to the target cellular protein. In some embodiments, the aptamer is a peptide aptamer. In other embodiments, the aptamer is a polynucleotide aptamer. In some embodiments, the binding moiety comprises a ligand that binds to a site on a target cellular protein. In some embodiments, one or more of the plurality of binding moiety-oligonucleotide conjugates targets an intracellular protein. In some embodiments, one or more of the plurality of binding moiety-oligonucleotide conjugates targets a nuclear protein. In some embodiments, one or more of the plurality of binding moiety-oligonucleotide conjugates targets a cytoplasmic protein. In some embodiments, one or more of the plurality of binding moiety-oligonucleotide conjugates targets a protein on the cell surface. In some embodiments, the step of incubating comprises incubating a population of cells with the population of binding moiety-oligonucleotide conjugates, and the method further comprises distributing single cells of the population of cells into single-cell analysis compartments. In some embodiments, the cells are permeabilized. In some embodiments, the step of distributing single cells is performed after the incubating step and the method comprises at least one washing step prior to an amplification step. In some embodiments, the single cell analysis compartments are droplets, microwells, or chambers of a microfluidic device. In some embodiments, the step of amplifying comprises incorporating a cellular identification sequence, a unique molecular identifier (UMI) sequence, and/or a sample identification sequence during amplification. In some embodiments, the method further comprises quantifying the levels of RNA transcripts in the single cell. In some embodiments, the method further comprises massively parallel sequencing for analysis of transposase-accessible chromatin in the single cell, HiC analysis, whole genome sequencing, mitochondrial DNA sequencing, methylation profiling, haplotype analysis, and CRISPR RNA sequencing.

In a further aspect, described herein is a method of quantifying the levels of a plurality of cellular proteins present in a cell, the method comprising (a) incubating a plurality of cells with a population of binding moiety-oligonucleotide conjugates comprising a plurality of conjugates in which each conjugate comprises a target binding moiety that specifically binds to a cellular protein to be quantified conjugated to an oligonucleotide that comprises a target-binding-moiety barcode sequence, said target-binding-moiety barcode sequence differing in sequence from target-binding-moiety barcode sequences contained in oligonucleotide components of conjugates that comprise different target binding moieties, wherein the oligonucleotide components of the conjugates are coated with a nucleic acid binding protein; (b) distributing subpopulations of cells of the population into compartments; (c) incorporating a cellular identification sequence during an amplification step performed on nucleic acids from the each of the subpopulations of cells of (b), wherein the cellular identification sequence for each subpopulation of (b) differs from the cellular identification sequence of other subpopulations of (b) distributed to other compartments; (d) pooling the subpopulations to obtain a pooled population of cells; (e) distributing subpopulations of the pooled population of (d) into compartments; (f) incorporating a cellular identification sequence during an amplification step performed on nucleic acids from each of the subpopulations of (e), wherein the cellular identification sequence for each subpopulation (e) differs from the cellular identification sequence of other subpopulations distributed to other compartments in step (e); and wherein steps (d)-(f) are optionally repeated; and (g) quantifying the level of barcode sequences for each binding moiety associated with the cell in the amplified product, thereby quantifying the level of each of the cellular proteins bound to the target binding moiety. In some embodiments, the plurality of cells of (a) are permeabilized cells. In some embodiments, the method further comprises quantifying the levels of RNA transcripts in the single cell. In some embodiments, the method further comprises massively parallel sequencing for analysis of transposase-accessible chromatin in the single cell, HiC analysis, whole genome sequencing, mitochondrial DNA sequencing, methylation profiling, haplotype analysis, and CRISPR sgRNA sequencing. In some embodiments, one or more of the plurality of binding moiety-oligonucleotide conjugates targets a protein on surface of the cell. In some embodiments, one or more of the plurality of binding moiety-oligonucleotide conjugates targets an intracellular protein. In some embodiments, one or more of the plurality of binding moiety-oligonucleotide conjugates targets a nuclear protein. In some embodiments, the nucleic acid binding protein preferentially binds to single stranded DNA. In some embodiments, the nucleic acid binding protein is Escherichia coli SSB or T4 gene 32 protein.

In a further aspect, the disclosure provides a method of quantifying the level of a target molecule in a single cell, the method comprising incubating the single cell with an oligonucleotide conjugate comprising a binding moiety that specifically binds the target molecule conjugated to an oligonucleotide comprising a barcode sequence that identifies the target molecule, wherein the oligonucleotide is coated with a nucleic acid binding protein; and quantifying the level of binding moiety bound to target molecule. In some embodiments, the nucleic acid binding protein is a sequence non-specific nucleic acid binding protein, such as a single-stranded nucleic acid binding protein. In some embodiments, the nucleic acid binding protein is Escherichia coli SSB or T4 gp32. In some embodiments, the target molecule is a protein. In some embodiments, the target molecule is an intracellular protein. In some embodiments, the binding moiety is an antibody. In some embodiments, the binding moiety is an aptamer or ligand. In some embodiments, the step of quantifying comprises amplifying a region of the oligonucleotide. In some embodiments, the step of quantifying comprises quantitative PCR and/or massively parallel sequencing. In some embodiments, the oligonucleotide comprises a detectable label or comprises a region that specifically hybridizes to a complementary oligonucleotide that comprises a detectable label, such as a fluorescent label. In some embodiments, quantification comprises detecting the level of a signal generated from the detectable label.

In a further aspect, described herein is a method of quantifying a target nucleic acid molecule in a cell, the method comprising hybridizing an oligonucleotide specific for the target nucleic acid to the target nucleic acid molecule, wherein the oligonucleotide is coated with a nucleic acid binding protein; and quantifying the amount of oligonucleotide hybridized to the target molecule. In some embodiments, the nucleic acid binding protein is a sequence non-specific nucleic acid binding protein, such as a nucleic acid binding protein that preferentially binds single-stranded DNA. In some embodiments, the nucleic acid binding protein is Escherichia coli SSB or T4 gp32. In some embodiments, the oligonucleotide comprises a detectable label, such as a fluorescent label. In some embodiments, quantifying the amount of oligonucleotide hybridized to the target molecule comprises detecting the level of a signal generated from the detectable label. In some embodiments, quantifying comprises an amplification reaction.

In another aspect, the disclosure provides an oligonucleotide conjugated to a target-binding moiety that specifically binds to a target cellular molecule, wherein the oligonucleotide is coated with a nucleic acid binding protein, e.g., a single-stranded sequence non-specific nucleic acid binding protein such as Escherichia coli SSB or T4 gp32. In some embodiments, the target molecule is a protein and/or the binding moiety is an antibody. In some embodiments, the target molecule is an intracellular protein.

The disclosure additionally provides an oligonucleotide that specifically hybridizes to target nucleic acid, wherein the oligonucleotide is coated with a nucleic acid binding protein, e.g., a single-stranded sequence non-specific nucleic acid binding protein, such as Escherichia coli SSB or T4 gp32.

In a further aspect, the disclosure provides a kit comprising (i) a plurality of binding moiety-oligonucleotide conjugates, wherein each conjugate comprises a target binding moiety that specifically binds to a cellular protein, wherein the oligonucleotide comprises an identifier sequence specific to the target binding moiety, and the identifier sequence differs in sequence from the identifier sequences conjugated to binding moieties that specifically bind to other cellular proteins; and (ii) at least one nucleic acid binding protein. In some embodiments, the at least one nucleic acid binding protein is a sequence non-specific nucleic acid binding protein, such as a single stranded DNA binding (SSB) protein. In some embodiments, the nucleic acid binding protein is Escherichia coli SSB or T4 gp32. In some embodiments, the kit further comprises a buffer for binding of the at least one sequence non-specific nucleic acid binding protein to the oligonucleotide and/or a permeabilization buffer and/or primers to amplify the oligo and/or sequencing adapters.

In other aspects, a kit as described herein comprises a plurality of binding moiety-oligonucleotide conjugates, wherein each conjugate comprises a target binding moiety that specifically binds to an intracellular protein, wherein the oligonucleotide comprises a barcode sequence that identifies the target binding moiety, and the identifier sequence differs in sequence from the identifier sequences conjugated to target binding moieties that specifically bind to other intracellular proteins, wherein the oligonucleotide component is coated with a nucleic acid binding protein, such as a sequence non-specific nucleic acid binding protein, e.g., a single-stranded sequence non-specific DNA binding protein. In some embodiments, the nucleic acid binding protein is Escherichia coli SSB or T4 gp32.

### BRIEF DESCRIPTION OF THE DRAWINGS

FIG. 1A-G. Addition of single stranded DNA binding protein (SSB) to oligonucleotide-conjugated antibody prior to staining enables specific staining of nuclear proteins. A) Schematic showing that incubation of oligo-conjugated antibodies (blue and purple) with SSBs (grey) results in binding and blocking of the conjugated oligos. This allows the antibodies to penetrate permeabilized nuclei and stain nuclear target proteins with low background. B) Flow cytometry plot of HEK293T cells expressing nuclear-localized GFP and stained with an anti-GFP antibody linked to an 80 bp single stranded DNA oligo with 3′-Cy5 modification. C) Flow cytometry plot of HEK293T cells expressing cytosolic GFP and stained with an anti-GFP antibody linked to a 100 bp single stranded DNA oligo with 3′-Cy5 modification. D) Staining of K562 cells and mouse ESCs for endogenous GATA1 protein using an antibody conjugated to an oligo with 3′-Cy5. E) Sorting of cells expressing low, mid, or high levels of GFP that have been stained with an anti-GFP oligo-conjugated antibody as in (B); and quantitative PCR for the conjugated oligo from equal cell numbers of sorted populations (F); (G)“NEAT-seq”: NEAT-seq fixation, permeabilization, and staining conditions using oligo-antibodies pre-incubated with EcoSSB. “Dextran sulfate block”: NEAT-seq fixation and permeabilization conditions with inCITE-seq staining conditions (i.e with dextran sulfate blocking agent). “inCITE-seq”: inCITE-seq fixation, permeabilization, and staining conditions. Spearman correlation is shown.

FIG. 2A-D. Measurement of intranuclear protein abundance in single cells using oligo-barcoded antibodies can be combined with other single cell genomic measurements. A) An example application of this staining method to profile intracellular protein abundance along with chromatin accessibility in single cells using the 10× Genomics Chromium platform. Individual cells are encapsulated in emulsion droplets along with gel beads coated with barcoded oligos as shown. Conjugation of antibodies with complementary ssDNA oligos allows extension and amplification of PCR products containing the necessary components to be able to measure the abundance of specific target proteins in individual cells via high-throughput sequencing. B) The fraction of log-transformed antibody-derived oligo reads that correspond to the indicated antibody target in mouse ESCs vs human K562 cells from a scATAC-seq experiment performed on a 1:1 mixture of K562 and mouse ESCs stained with oligo-conjugated antibodies against TFs. C) Pooled TSS enrichment score for the scATAC-seq library from the experiment described in (B). D) Fragment length distribution of the scATAC-seq library.

FIG. 3 depicts oligonucleotide employed for combined assays comprising an antibody-oligonucleotide conjugate assay to quantify protein as described herein, ATAC-seq and RNA-seq.

FIG. 4A-C provides data illustrating measurement of nuclear protein abundance using oligonucleotide-barcoded antibodies in combination with chromatin accessibility and gene expression profiling in single cells. Panel A shows the median genes detected per cell as a function of sequencing depth as measured by mean reads per cell in the RNA-seq library. Panel B shows the TSS enrichment score for the scATAC-seq library. Panel C shows the distribution of the antibody-derived oligo read counts (centered log ratio normalized) for OCT4 in human K562 ESCs (left peak) and mouse ESCs (right peak); and for GATA1 in human cells (right peak) and mouse cells (left peak).

FIG. 5A-C: Profiling of CD4 memory T cells using NEAT-seq reveals translational regulation of GATA3. A) Log2-transformed, NPC-normalized ADT counts for each TF separated by scATAC-seq cluster for cells stained with antibody concentration 1 and antibody concentration 2 (see methods) B) Scatterplot of loge-transformed, normalized RNA vs ADT counts for GATA3 with cutoffs shown for high RNA, high protein, and low protein indicated. C) Differentially expressed genes between cells with high RNA and high protein vs high RNA and low protein for GATA3.

FIG. 6A-B: NEAT-seq performed on primary human bone marrow mononuclear cells (BMIVICs). A) Annotation of BMMC subsets clustered using scATAC-seq and scRNA-seq data after removing cell doublets and contaminating peripheral blood mononuclear cells. B) Distribution of protein levels in cells from each BMMC subset for the indicated transcription factor, as measured by oligo conjugated antibodies that were pre-incubated with SSB prior to staining. The x-axis plots log-transformed values for the number of sequencing reads mapping to the indicated antibody barcode normalized to the reads mapping to a housekeeping antibody barcode (targeting the nuclear pore complex.

### DETAILED DESCRIPTION

### Terminology

A “polynucleotide” or “nucleic acid” includes any form of RNA or DNA, including, for example, genomic DNA; complementary DNA (cDNA); DNA molecules produced by amplification; or synthetically produced DNA or RNA molecules. The terms include chimeric molecules and molecules comprising non-standard bases, modifications, or nucleotide analogs. For example, an oligonucleotide may contain naturally occurring nucleotides and/or analogs thereof. Polynucleotides may be single-stranded or double-stranded.

A “cellular polypeptide” or “cellular protein” is an intracellular, e.g., cytoplasmic or nuclear; membrane-associated; or extracellular protein produced by a cell.

A “target binding moiety” refers to any molecule that specifically binds a cellular target of interest, e.g., a protein. Such moieties include, but are not limited to antibodies, antibody mimetics, nucleic acid and peptide aptamers, ligands that bind to certain sites on proteins, e.g. ligands that bind to receptor proteins, lectins, lipids, glycolipids, polysaccharides, or synthetic ligands, that specifically bind to a cellular target, e.g., a target protein, for quantification of the level of the target protein in the cell. A “target binding moiety” also includes binding moieties that bind to the same cellular protein, but at different sites. The target binding moiety typically binds to the cellular target of interest via noncovalent binding interactions. The target binding moiety typically binds a cellular protein, but in some embodiments, may target other cellular molecules, such as a carbohydrate or glycolipid.

A “compartment” as used herein in the context of distribution of cells, refers to any partially or fully enclosed space that separates single cells, or pools of cells, from another. Thus, a compartment can include microwells, droplets, micropores, microfluidic chambers, and the like.

As used herein, the terms “a”, “an”, and “the” can refer to one or more unless specifically noted otherwise.

### Oligonucleotide-Target Binding Moiety Conjugates

Described herein are compositions and assay methods employing oligonucleotide-target binding moiety conjugates to quantify cellular molecules, preferably proteins, including intracellular proteins, such as cytoplasmic or nuclear proteins. As further described herein, coating of the oligonucleotide component with a nucleic acid binding protein, such as a sequence non-specific binding protein, to block non-specific binding of the charged oligonucleotide provides the ability to effectively quantify the target molecules.

### Binding Moiety

The binding moiety can be any molecule that specifically binds to a cellular target such that the amount of the target of interest present in the cell can be determined. The term “specific binding” refers to the ability of a binding moiety to preferentially bind to a particular cellular target when incubated with a permeabilized cell such that the level of the cellular target can be quantified, i.e., a specific binding interaction can discriminate between target molecules and non-target molecules such that the amount of the specific target present in the cell can be determined. For example, the binding of a binding moiety to its target may be from 10-fold to 10,000-fold greater compared to its binding to a non-target cellular molecule.

In some embodiments, the binding moiety is an antibody. The term “antibody” encompasses full-length antibody formats, e.g., IgG, and functional fragments of antibodies that bind the target antigen, including multimeric and monomeric forms. The term encompasses polyclonal and monoclonal antibody preparations, and chimeric antibodies or other engineered antibodies. “Antibody” thus also refers to binding formats including diabodies, triabodies, tetrameric forms, single domain antibodies and the like. A functional fragment can be a portion of an antibody such as a F(ab′)2, Fab′, Fab, Fv, or can be an engineered binding fragments, such as an scFV. In some embodiments, the binding moiety may be an antibody mimetic. Examples included fibronectin-scaffold based polypeptides such as adnectins and ankyrin repeat scaffolds such as DARPins.

In some embodiments, the binding moiety is a ligand that binds to a specific site to a target cellular molecule, e.g., target cellular protein, and includes ligands for cellular receptors, enzymes, or other proteins. The ligand may be a polypeptide molecule, small molecule, or any molecule that binds to a cognate cellular binding partner.

In some embodiments, the binding moiety can be a nucleic acid or peptide aptamer. Aptamers interact with their targets by recognizing a specific three-dimensional structure. Peptide aptamers are composed of a short variable peptide loop attached at both ends to a protein scaffold such as the bacterial protein thioredoxin-A. A peptide aptamer specific to a target of interest may be selected using any method known by the skilled person such as the yeast two-hybrid system or phage display. Peptide aptamers may be produced by chemical synthesis or recombinantly produced.

In some embodiments, the aptamer is a nucleic acid aptamer. Nucleic acid aptamers are a class of small nucleic acid ligands that are composed of RNA or single-stranded DNA oligonucleotides folded into a three-dimensional structure that have high specificity and affinity for their targets. For example, Systematic Evolution of Ligands by Exponential enrichment (SELEX) technology can be used to obtain aptamers specific to a particular molecular target. Nucleic acid aptamers can be produced by as chemical synthesis or in vitro transcription for RNA aptamers. Nucleic acid aptamers include DNA aptamers, RNA aptamers, ×NA aptamers (nucleic acid aptamer comprising xeno nucleotides) and L-RNA aptamers.

Suitable target binding moieties that bind to a cellular molecule of interest are also described, e.g., in US Patent Application Publication Nos. 20200087707 and 20200385780.

### Oligonucleotide Component

In some embodiments, an oligonucleotide component of a target binding moiety-oligonucleotide conjugate to quantify cellular targets as described herein comprises an identifier sequence specific for a binding moiety, i.e., the barcode sequence, which differs in sequence from the barcode region of oligonucleotides conjugated to target binding moieties that bind to different targets. The oligonucleotide may be double or single-stranded and in some embodiments, may comprise single-stranded and double stranded regions.

The barcode regions may vary in length, e.g., depending on the number of target binding moieties in the populations of conjugates used to quantify cellular targets. In certain embodiments, the barcode region can have a length, for example, of 4, 5, 6, 7, 8, 9, 10, 11, 12, 13, 14, 15 nucleotides, or longer.

The oligonucleotides may be DNA, RNA, a combination, or may comprise one or more non-naturally occurring nucleotides, nucleotide analogs, or and/or chemical modifications. Non-naturally occurring nucleotides and/or nucleotide analogs can be modified at the ribose, phosphate, and/or base moiety. Examples of modified base moieties include, but are not limited to: 5-fluorouracil, 5-bromouracil, 5-chlorouracil, 5-iodouracil,hypoxanthine, xanthine, acetylcytosine, 5-(carboxyhydroxylmethyl) uraci1,5-carboxymethylaminomethyl-2-thiouridine,5-carboxymethylaminomethyluracil, dihydrouracil,beta-D-galactosylqueosine, inosine, N.about.6-sopentenyladenine,1-methylguanine, 1-methylinosine, 2,2-dimethylguanine, 2-methyladenine,2-methylguanine, 3-methyl cytosine, 5-methyl cytosine, N6-adenine,7-methylguanine, 5-methylaminomethyluracil,methoxyarninomethyl-2-thiouracil, beta-D-mannosylqueosine,5′-methoxycarboxymethyluracil, 5-methoxyuraci1,2-methylthi o-N6-isopentenyladenine, uracil-5-oxyacetic acid,pseudouracil, queosine, 2-thiocytosine, 5-methyl-2-thiouraci1,2-thiouracil, 4-thiouracil, 5-methyluracil, uracil-5-oxyacetic acidmethylester, uracil-S-oxyacetic acid, 5-methyl-2-thiouraci1,3-(3-amino-3-N-2-carb oxypropyl) uracil, 2,6-diaminopurine andbiotinylated analogs, amongst others. Examples of modified sugar moieties include, but are not limited to, arabinose, 2-fluoroarabinose, xylose, and hexose, or a modified component of the phosphate backbone, such as a phosphorothioate, a phosphorodithioate, a phosphoramidothioate, a phosphoramidate, a phosphordiamidate, a methylphosphonate, an alkylphosphotriester, or a formacetal or analog thereof. In some embodiments, an oligonucleotide can comprise one or more ribonucleotides and one or more deoxyribonucleotides. In some embodiments the oligonucleotide may comprise a boranophosphate linkage, a locked nucleic acid (LNA) nucleotide, a peptide nucleic acid (PNA), or bridged nucleic acids (BNA).

The oligonucleotide may comprise regions in addition to the barcode sequence that include, but are not limited to, primer binding sites for sequencing primers, primer binding sites for subsequent amplification, and a unique molecular identifier sequence (UMI) specific for the molecule. In some embodiments, the oligonucleotide may comprise a cell identification region that identifies the cell in which cellular targets, e.g., cellular proteins, are quantified. Other regions that can be incorporated into an oligonucleotide include adaptor sequences. The positions of the elements of the oligonucleotide need not occur in a specific order, for example, a UMI may be positioned at the 5′ or 3′ end of the barcode sequence. In some embodiments, an oligonucleotide further comprises a sample indexing sequence (also referred to as a sample identifier sequence), which allows identification of the sample from which the cell is obtained. As understood in the art, a cell indexing sequence or sample indexing sequence can be added to the oligonucleotide in an amplification reaction after incubation of the conjugate with individual cells.

In other embodiments, the oligonucleotide may comprise an identifier sequence compatible with another single-cell analysis assay, such as a chromatin accessibility ATAC-Seq assay, or RNA expression assays as further described below.

### Conjugation of Oligonucleotide to Target Binding Moiety

Various methods can be employed to conjugate oligonucleotides target binding molecules. The oligonucleotide component and target binding component may be chemically conjugated (e.g., via a linker) or conjugated such that the oligonucleotide can be removed from the protein-binding molecule via cleavage. For example, heterobifunctional cross-linkers, such as succinimidyl4-hydrazinonicotinate acetone hydrazone (SANH) and succinimidyl 4-(N-maleimidomethyl)cyclohexane-1-carboxylate (SMCC are often used to introduce a bridge between an oligonucleotide and an antibody. Commercial kits are also available for the production of oligonucleotide conjugates (e.g., Abcam antibody-oligonucleotide conjugation kit). Other conjugation reactions include click reactions.

In some embodiments, a streptavidin-biotin interaction may be employed to link oligonucleotides to target binding moieties. In certain embodiments, the conjugate may include a disulfide link at the 5′ end of the oligonucleotide to allow release of the oligonucleotide using reducing agents.

In some embodiments, an oligonucleotide may be attached to a target binding moiety through the sequential addition of a dibenzocyclooctyne (DBCO) moiety and an azide-modified oligonucleotide. In other embodiments, antibodies may be chemically cross-linked to a substrate that contains free amino or carboxyl groups using glutaraldehyde or carbodiimides as cross-linker agents.

### Nucleic Acid Binding Proteins

In the present disclosure, oligonucleotide components are incubated with a nucleic acid binding protein to block or reduce non-specific binding. An oligonucleotide complexed with a nucleic acid binding protein is referred to herein as an oligonucleotide “coated” with the binding protein. In some embodiments, the nucleic acid binding protein is a sequence non-specific nucleic acid binding protein.

### Sequence Non-Specific Nucleic Acid Binding Proteins

In some embodiments, the nucleic acid binding protein is a single-stranded nucleic acid binding protein involved in replication. Thus, in some embodiments, the nucleic acid binding protein is a single-stranded DNA binding protein (SSB). Most SSBs preferentially bind DNA, i.e., binding to RNA is much weaker (see, e.g., Ashton et al, BMC Mot Biol. 14:9, 2013), however SSBs from hyperthermophilic organism have been described (see, e.g., Morten et al, Extremophiles 21:369-379, 2018) that demonstrate essentially the same binding properties for ssRNA or ssDNA, which may also be used for coating oligonucleotides to block or reduce non-specific binding as described herein. SSBs have the general property of preferentially binding to single-stranded nucleic acid compared to double-stranded nucleic acid; i.e., they bind more strongly to single-stranded vs double-stranded nucleic acids e.g., have an affinity for single-stranded DNA that is at least two-fold, or at least five-fold, greater than the affinity for double-stranded DNA. SSB binding to nucleic acids is not dependent the presence of specific sequences in the target. Structures of SSBs that mediate binding have been described (see, Ashton et al, 2013 and Morten et al, 2018, supra). Single-stranded binding proteins from any source, including for example prokaryotes, e.g., bacteria, eukaryotes, Archaea, and viruses may be employed to coat oligonucleotides as described herein. Examples of single stranded binding proteins include, but are not limited to, E coli SSB, T4 gene 32 (T4 gp32) protein, Tth RecA, human replication protein-A (RPA), herpes simplex virus 1CP8 protein, vaccinia virus single strand binding protein, and ET SSB, a thermostable single-stranded DNA binding protein. See also, Chase et al, Ann. Rev. Biochem. 55:103-36, 1986; Coleman et al, CRC Critical Reviews in Biochemistry 7:247-289, 1980) and U.S. Pat. No. 5,773,257.

In some embodiments, suitable bacterial SSB homologs include those from Listeria innocua, Thermus aquaticus, Thermus thermophiles, M smegmatis, and D. radiodurans. Suitable Archaeal ssDNA-binding proteins include, but are not restricted to SSB from Methanococcus jannaschii, Methanobacter theromoautotrophicum, Archaeoglobus fulgidus, Sulfolobus Solfataricus P2 (SSOB), and Thermococcus kodakarensis.

Viral single-stranded DNA-binding proteins include, but are not restricted to viral SSB, such as adenovirus-encoded DNA binding protein, EBV BALF2 protein, Herpes simplex virus type 1 single-strand DNA binding protein ICP8, T4 gp32, T4 gene 44/62 protein, T7 SSB, coliphage N4 SSB, adenovirus DNA binding protein (Ad DBP or Ad SSB), and calf thymus unwinding protein (UP1).

In some embodiments, a single-stranded sequence non-specific nucleic acid binding protein has a dissociation constant for binding single-stranded DNA of ˜10 μM or lower. In some embodiments, the dissociation constant is about 1 μM or lower. In some embodiments, the dissociation constant is in the nM range, e.g., the dissociation constant is less than 100 nM.

In some embodiments, the sequence non-specific nucleic acid binding protein is a double-stranded nucleic acid binding protein. Examples include bacterial and archaeal DNA packaging proteins, e.g., Archael 7kDA Sso7d and Sac7d family of binding proteins (see, e.g., Kalichuk et al, Sci. Reports 6:37274; DOI: 10.1038/srep37274, 2016); Dps proteins found in a variety of bacteria and archaea that bind to DNA non-specifically in response to oxidative stress to prevent DNA damage (J. Applied Microbiol. 110:375-386, 2010; and HMG proteins (Murphy & Churchill, Structure 8:$83-R89, 2000). In some embodiments, the nucleic acid binding protein has a dissociation constant of ˜10 μM or lower. In some embodiments, the dissociation constant is about 1 μM or lower. In some embodiments, the dissociation constant is in the nM range, e.g., the dissociation constant is less than 100 nM.

In some embodiments, an oligonucleotide-target binding moiety conjugate can be coated with a nucleic acid binding protein that exhibits sequence specificity. In some embodiments, such a nucleic acid binding protein is a sequence-specific DNA binding protein. In some embodiments, the sequence-specific binding protein has a dissociation constant that is at least 100-fold greater for a nucleic acid molecule comprising the nucleic acid binding protein recognition sequence compared to another nucleic acid molecule of the same composition, but a different nucleotide sequence, assayed under the same conditions. Thus, for example, in some embodiments, oligonucleotide components may include binding sites for a sequence-specific nucleic acid binding protein.

Coating of Oligonucleotides with Nucleic Acid Binding Protein

An oligonucleotide is “coated” with the nucleic acid binding protein, e.g., a sequence non-specific nucleic acid binding protein, by incubating the protein with the oligonucleotide-target binding moiety conjugate. This can be performed prior to incubating conjugates with cells or can be performed at the same time. In some embodiments, one or more coated conjugates may be tested for blocking activity by incubating cells, e.g., permeabilized cells, with conjugates, e.g., that target intracellular proteins, labeled with a detectable label, such as a fluorescent label, to assess background levels of cell staining with conjugates coated with nucleic acid binding protein(s) compared to the uncoated control conjugate. In typical embodiments, non-specific binding is reduced by at least 90% using the coated oligonucleotide conjugate compared to the control oligonucleotide without reducing signal from binding of the conjugate to the target polypeptide. The nucleic acid binding protein is incubated in stoichiometric excess relative to the oligonucleotide-target binding moiety conjugate. The minimum concentration of binding protein to ensure that most conjugates (for a given concentration) are coated can be estimated based on the dissociation constant of the nucleic acid binding protein.

In typical embodiments, the oligonucleotide may be coated with one nucleic acid binding protein, e.g., an SSB; however, in some embodiments, the oligonucleotide may be coated with a mixture of nucleic acid binding proteins that comprises more than one binding protein. For example, the mixture may comprise both a single-stranded DNA binding protein and a double-stranded DNA binding protein. Such a mixture may be employed, for example, in instances in which an oligonucleotide comprises both single-stranded and double-stranded regions.

### Assay of Cellular Target Molecules

Although use of a nucleic acid binding protein, e.g., a sequence non-specific nucleic acid binding protein, to block non-specific binding of an oligonucleotide-target binding moiety conjugate is largely described herein in the context of an oligonucleotide that contains a target binding moiety-specific barcode for quantification using massively parallel sequencing, one of skill understands that this blocking technique to reduce non-specific binding can be employed in any embodiment in which a target binding moiety, such as an antibody, is joined to an oligonucleotide to analyze cells. For example, in some embodiments a single cell may be incubated with a conjugate comprising an oligonucleotide (coated with a binding protein such as a sequence non-specific nucleic acid binding protein) conjugated to a target binding moiety, e.g., an antibody, that binds a target molecule in the cell, such as a target protein molecule. In some embodiments, following incubation, the oligonucleotide can be amplified to quantify the level of binding moiety bound to its target. In some embodiments, the oligonucleotide component further comprises a cell and/or sample identification sequence. In some embodiments, the level of binding moiety is assessed by measuring hybridization of the oligonucleotide to a complementary oligonucleotide. In some embodiments, the oligonucleotide and/or complementary oligonucleotide are labeled with a detectable label, e.g., a fluorescent label, and the level of the binding moiety is assessed by quantifying a signal generated from the detectable label.

In some embodiments, an oligonucleotide that specifically hybridizes to a target nucleic acid present in a cell, e.g., an mRNA for a target gene of interest, is coated with a nucleic acid binding protein, e.g., a sequence non-specific nucleic acid binding protein, such as a single-stranded DNA binding protein such as SSB or T4 gp32 for hybridization to the target nucleic acid, e.g., in a cell or tissue. Not to be bound by theory, coating the oligonucleotide as described herein is thought to decrease background binding of oligonucleotide to cellular components and thus provides increased assay sensitivity. In some embodiments, the assay is an in situ hybridization, such as fluorescent in situ hybridization. In some embodiments, the oligonucleotide is coated with the nucleic acid binding protein prior to hybridization. In some embodiments, the oligonucleotide is coated during hybridization.

### Protein Assay Methods

In some embodiments, methods as described herein provide the ability to quantify cellular molecules of interest, e.g., cellular proteins, including intracellular proteins, in a single cell. A cell to be analyzed is incubated with a plurality of oligonucleotide/target binding moiety conjugates. In some embodiments, the cells are permeabilized. In some embodiments, cells are distributed as single cells to separate compartments, e.g., based on Poisson distribution, after incubation with the plurality of conjugates. In some embodiments, cells may be distributed to separate compartments prior to incubation with the plurality of conjugates. One of skill understands that the method can further comprise one or more wash steps, e.g., following incubation with conjugates. An amplification reaction is then typically performed following incubation to generate amplified products as a library for sequencing. As used herein, “amplification” of a nucleic acid sequence has its usual meaning, and refers to in vitro techniques for enzymatically increasing the number of copies of a target sequence. Amplification methods include both asymmetric methods in which the predominant product is single-stranded and conventional methods in which the predominant product is double-stranded. In typical embodiments PCR is used to obtain amplified products.

A cell identifier sequence and/or a sample identifier sequence can be incorporated into amplification products to identify the sample/cell and the amplified products processed for massively parallel sequencing to identify and quantify the level of the target binding moiety-specific barcode associated with the single cell. This provides a quantitative protein expression profile for the single cell.

In some embodiments, single-cell combinatorial indexing using split and pool techniques to label the contents of a single cell with a cellular identification sequence is employed. For example, in this method cells are distributed to a compartment, such as a well, following incubation with a population of target binding moiety-oligonucleotide conjugates. During processing, a cell-specific identification sequence can be introduced into the product amplified from the oligonucleotide component. The contents of the compartments, e.g., wells, are then pooled and redistributed to a second set of wells, in which a second cellular identification sequence is introduced. In some embodiments, this procedure can be repeated to introduce a third cellular identification sequence, or more, if desired. The redistribution of cells through a unique combination of wells allows the identification of a cell by the unique barcode combination they receive. For example, to generate single cell libraries through split and pool combinatorial indexing, fixed and permeabilized cells are distributed into wells of a plate (e.g., 96-well, 384-well, or microwell plates) containing multiple cells per well (e.g., −50-1000) and a first barcode is added in an amplification reaction. All cells across wells are then repeatedly pooled and redistributed to undergo successive rounds of barcoding so that individual cells will have a unique combination of cell indexing sequences.

In some embodiments, the method further comprises performing additional analyses on the single cell. Various single cell assays can be used for evaluating a sequence-based single cell parameter. Exemplary assays include RNA sequencing, including, but not limited to, sequencing of mRNA, and other RNA populations of interest such as miRNA, snRNA, lncRNA and the like; and genomic DNA sequencing, including, but not limited to, haplotyping and phase determination, genotyping, intron and/or exon sequences, HiC, DNA methylation, CRISPR gRNA screens, and whole genome sequencing. In some embodiments, target genomic nucleic acids can be analyzed by ATAC-Seq, e.g., by capturing mosaic Tn5 sequences.

In some embodiments, the additional assay evaluates RNA expression using single cell RNA sequencing (RNA-Seq), which is described, e.g., by Tang et al., Nat. Methods 6:377-382, 2009; Ramskolod et al., Nat. Biotechnology 30:777-782, 2012; Macosko et al., Cell 161: 1202-1214, 2015; WO2016/040476; Klein et al., Cell 161:1187-1201, 2015; WO2016168584; Zheng, et al., Nature Biotechnology 34:303-311, 2016; Zheng, et al., Nat. Commun. 8: Article number 14049, 2017; WO 2014210353; Zilionis, et al., Nat Protoc. 12:44-73, 2017; Cao et al., Science 357:661-667, 2017; and Rosenberg et al., 2017, “Scaling single cell transcriptomics through split pool barcoding” bioRxiv preprint Feb. 2, 2017. Both unbiased and targeted approaches may be employed. Thus, for example, RNA capture oligonucleotides, e.g., comprising a polydT tract comprising cell identification barcodes may be attached to a substrate in a compartment containing the single cell and conjugate molecules of the present disclosure. Poly(A)+ RNA can then be reverse transcribed and prepared for sequencing.

In some embodiments, the method further comprises an ATAC-Seq analysis, of the single cell, which assesses chromatin accessibility (see, e.g., Buenrostro et al., Nat. Methods 10: 1213-8; 2013; Cusanovich, et al.,. Science 348:910-4. 2015; Qu et al., Cell Systems 1:51-61, 2015; Chen et al., Nat. Methods 13:1013-1020, 2016). Thus, in some illustrative embodiments, the method comprises incubating oligonucleotide-target binding moiety conjugates, e.g., oligonucleotide-antibody conjugates, with cells followed by incubating cells with reagents for ATAC-Seq, including a Tn5 transposase, distributing the cells into compartments, and capture of ATAC fragments, e.g., using a gel bead having attached thereto an oligonucleotide for ATAC-Seq analysis. Libraries can then be prepared for sequencing. In some embodiments, antibody incubation of the binding moiety conjugate with cells is performed after Tn5 transposition.

In further embodiments, whole genome sequencing can be performed, e.g., for haplotype analysis or phasing. Illustrative whole genome sequencing reactions for single cell genome sequence include multiple displacement amplification (MDA) and Multiple Annealing and Lopping Based Amplification Cycles (MALBAC) (see, e.g., Stepanauskas et al, Nat. Comm. 8: Article number 84, 2017; Zong et al, Science 338:1622-1626, 2012; Nin et al., Sci Rep 5:11415, 2015; Zhang et al, Nat. Commun 6:6822, 2015).

In some embodiments, quantitative analysis of proteins using the oligonucleotide-target binding moiety conjugates as described herein is performed concurrently with more than one additional single cell analysis assay, for example RNA-Seq and ATAC-Seq.

Single cell compartments may include droplets, microwells, microfluidic device chambers, micropores, and the like.

### Spatial Proteomics

In some embodiments, oligonucleotide-target binding moiety conjugates as described herein are employed for spatial proteomics to map binding interactions in tissues or cells. For example, a highly mutliplex cytometric imaging approach, CO-Detection by indEXing (CODEX) (see, e.g., Goltsev, et al, Cell 174, 968-981. e15, 2018; Black et al, Nat. Protoc. 16:3802-3825, 2021) can be employed to visualize antibody binding events using the target binding moiety-specific barcodes to provide multidimensional protein expression analysis and position data. For example, multiplex imaging methods can be used to evaluate cell types in a tissue specimen and to provide single cell spatial information. In some embodiments, a multi-antibody detection pool can be employed to identify spatial distributions of multiple cell types. In some embodiments, an oligonucleotide-target binding moiety comprises a detectable label, e.g., a fluorescent label. In some embodiments, the oligonucleotide comprises the label. In some embodiments, the target binding moiety comprises the detectable label. In some embodiments an oligonucleotide that hybridizes to the oligonucleotide component of the conjugate comprises a detectable label. In some embodiments, protein expression is quantified, e.g., by quantifying the signal generated from the detectable label. In some embodiments, protein is quantified by amplification and/or sequence analysis.

In some embodiments, spatial data obtained using oligonucleotide-antibody conjugates coated with nucleic acid binding proteins as described herein can be integrated with data from single cell gene expression profiling by scRNA-Seq. In some embodiments, techniques such as Slide-seq (Rodrigues et al., Science 363:1463-1467, 2019) can be employed in conjunction with protein quantification using oligonucleotide-target binding moiety conjugates coated with nucleic acid binding proteins as described herein. In some embodiments, protein expression can be mapped at the single cell levels within tissues by image analysis in combination with protein quantification. In some embodiments, the amount of protein in a cell or tissue is quantified. For example, in some embodiments, oligonucleotides in the oligonucleotide-target binding moiety conjugates are cleaved or released and annealed to spatially barcoded oligonucleotides attached to a slide. Extension of the annealed oligonucleotides to yield an oligonucleotide containing both protein target barcode and spatial barcode enables quantification of protein abundance at each spatial location on the slide by sequencing.

### Single Cells

Single cells from any source, including any plant, animal, or microorganism may be analyzed in accordance with the methods of the invention. In some embodiments, cells are eukaryotic cells, including, but not limited to, yeast and fungi cells, plant cells, avian cells, mammalian cells, and the like. In some embodiments, the cells are mammalian cells, e.g., human cells. In some embodiments, the cells are cancer cells, stem cells, neurological cells, peripheral blood mononuclear cells, lymphocytes, or cells from a cell line. In some embodiments, the cells are obtained from a tissue e.g., a human tissue. In some embodiments, the cells are obtained from a tumor, e.g., a human tumor. In some embodiments, single cells from transgenically modified organisms may be evaluated, e.g., for CRIPSPR-based screening.

### Kits

In a further aspect, the disclosure provides kits and reagents for quantification of cellular targets, e.g., cellular proteins. In some embodiments, a kit can comprise a plurality of target binding moiety-oligonucleotide conjugates as described herein; and reagents, such as a nucleic acid binding protein, such as sequence non-specific nucleic acid binding protein, e.g., an SSB protein or a T4 gp32 protein. In some embodiments, the kit may further comprise reagents such as a permeabilization buffer, primers to amplify the oligo and/or sequencing adapters.

In some embodiments, a kit comprises a plurality of target binding moiety-oligonucleotide conjugates that comprise one or more conjugates that contain a target binding moiety that specifically binds to an intracellular protein. In some embodiments, such a kit further comprises a nucleic acid binding protein, such as a sequence non-specific nucleic acid binding protein. In some embodiments, the kit comprises one or more conjugates that target a cell surface molecule such as a cell surface protein.

The following examples illustrate methods and compositions of the present disclosure. The disclosure is not limited to the particular embodiments employed in the examples.

### EXAMPLES

### Example 1. ssDNA Binding Protein-Coated Oligonucleotide-Antibody Conjugates

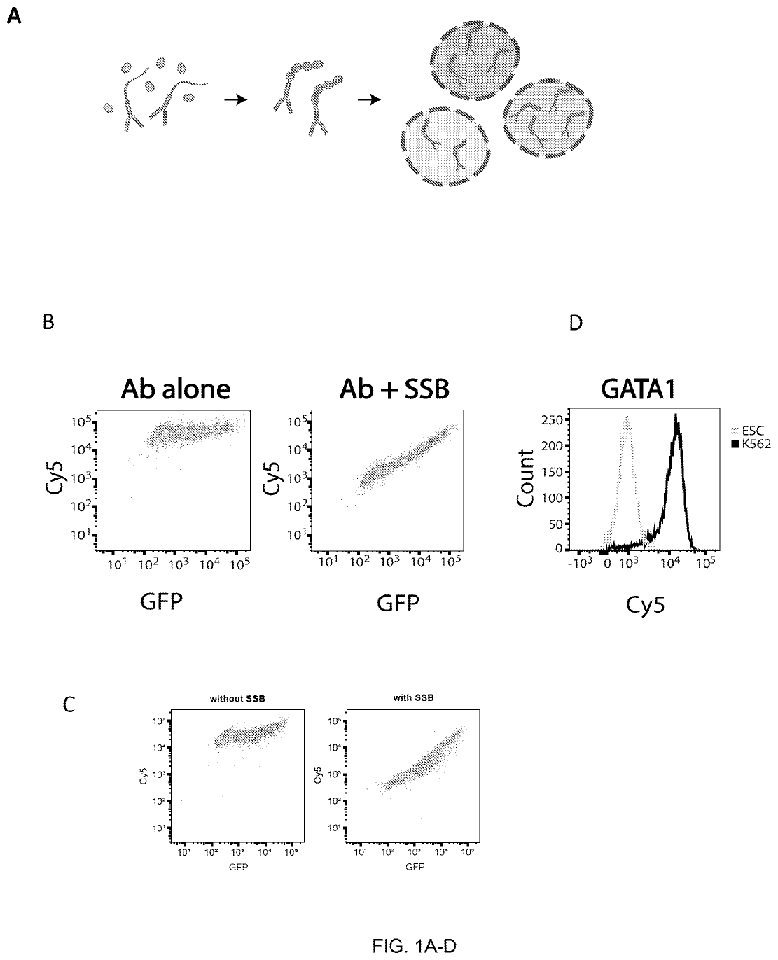

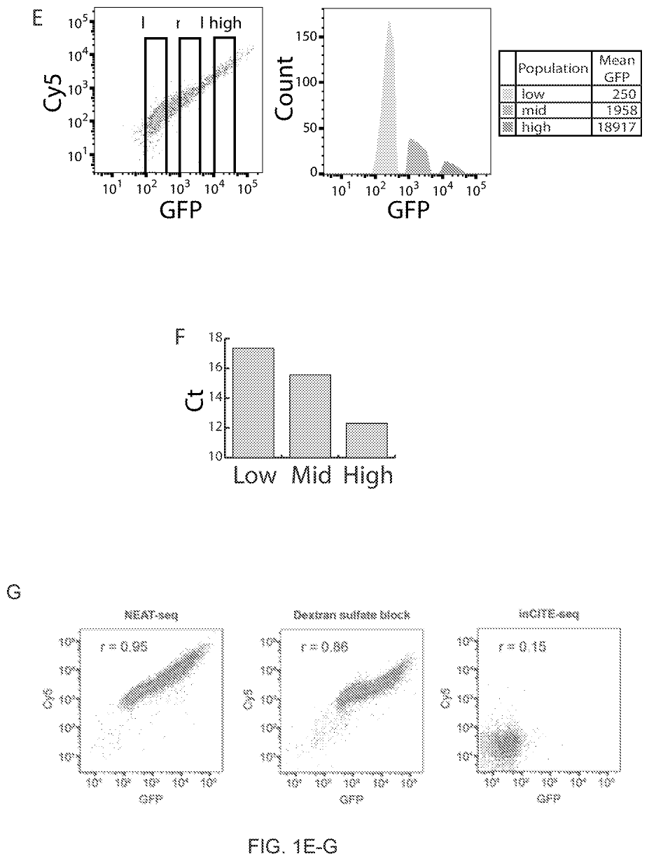

This example illustrates a method to block the negatively charged oligonucleotide conjugated to antibodies using sequence non-specific nucleic acid binding proteins, in this example, ssDNA binding proteins (SSBs), thus providing highly sensitive and specific staining of cytoplasmic and nuclear proteins. SSBs are a class of proteins that bind to and stabilize ssDNA and can facilitate cellular processes in which ssDNA is generated, such as during DNA replication. By pre-incubating purified SSBs with the oligo-conjugated antibodies, the SSBs bind to free ssDNA molecules and block non-specific binding of oligonucleotides to cellular material. Coating with the SSBs may also facilitate oligonucleotide penetration of the cell and/or nuclear membrane. The SSB-bound oligo-conjugated antibodies can then be used to stain permeabilized cells. An example of conjugate binding following incubation with permeabilized nuclei using typical staining procedures is provided in FIG. 1A. Notably, the role of SSBs in facilitating DNA replication means that the presence of bound SSBs on the antibody oligo is compatible with PCR amplification of the oligo required for downstream sequencing.

Antibody-oligonucleotide conjugates were prepared by initially conjugating streptavidin to the antibodies using an streptavidin-conjugation-kit (Abcam). Biotinylated oligonucleotide was then incubated overnight with the antibody at room temperature, and excess oligonucleotides removed.

Oligonucleotide-antibody conjugates were coated with SSB prior to incubation with cells. Coating of oligo-antibodies with SSB was performed by incubating the oligonucleotide-antibody conjugate with SSB (Promega) in NEB buffer 4 (New England Biolabs) for 30 mins at 37° C. The SSB is in stoichiometric excess relative to the oligo-antibody conjugate. Based on the dissociation constant of the SSB used, the minimum concentration of SSB required for a given concentration of oligo-antibody conjugate to ensure that most oligos are bound by SSB can be estimated.

As detailed below, the importance of using SSBs to block the conjugated oligos is demonstrated from our experiments using an anti-GFP antibody conjugated to oligos labeled with 3′ Cy5 to stain cells expressing a nuclear-localized GFP. The Cy5 fluorophore allows us to measure how accurately the antibody-oligo level reflects target protein (i.e GFP) levels via flow cytometry. In the absence of SSBs, high background staining with little correlation between antibody-oligo and GFP abundance is observed (FIG. 1B). However, pre-incubation of the antibody with SSBs results in lower background staining and high correlation between antibody-oligo and GFP levels. We further demonstrated that this staining procedure is sufficiently sensitive to detect endogenous expression of transcription factors. By staining for the transcription factor, GATA1, in a cell line that is negative (ESCs) and positive (K562s) for GATA1, we observed GATA1 staining specifically in K562 cells (FIG. 1D). To determine whether quantification of the oligonucleotide accurately reflects target protein levels, we sorted GFP-expressing cells that had been stained with the GFP antibody-oligo for populations expressing low, middle, and high levels of GFP and performed quantitative PCR for the conjugated antibody oligo in each sample (FIG. 1E). The approximately 8-fold difference in GFP levels between low and mid populations and 10-fold difference between mid and high populations observed based on GFP fluorescence are closely reflected in the difference in Ct values for amplification of the oligo (FIG. 1F). Together, these results show that oligo-conjugated antibody staining of nuclear target proteins accurately measures target protein abundance when SSBs are used to block the conjugated oligo. SSB can similarly improve specificity of cytosolic protein staining (FIG. 1C).

We also compared our EcoSSB staining to the staining protocol in inCITE-seq, which uses dextran sulfate to enable nuclear protein staining using oligo-antibodies¹⁹. In our hands, the inCITE-seq conditions resulted in a significant loss of GFP protein, perhaps due to the simultaneous fixation and permeabilization procedure (FIG. 1E). Using the fixation and permeabilization protocol that we developed and then staining with either our protocol (NEAT-seq) or inCITE-seq conditions (methods), we observed a stronger correlation between GFP abundance and antibody staining using our conditions (NEAT-seq r=0.95 vs inCITE-seq r=0.86; FIG. 1G).

### Example 2. Combined Assay: Compatibility with ATAC-Seq

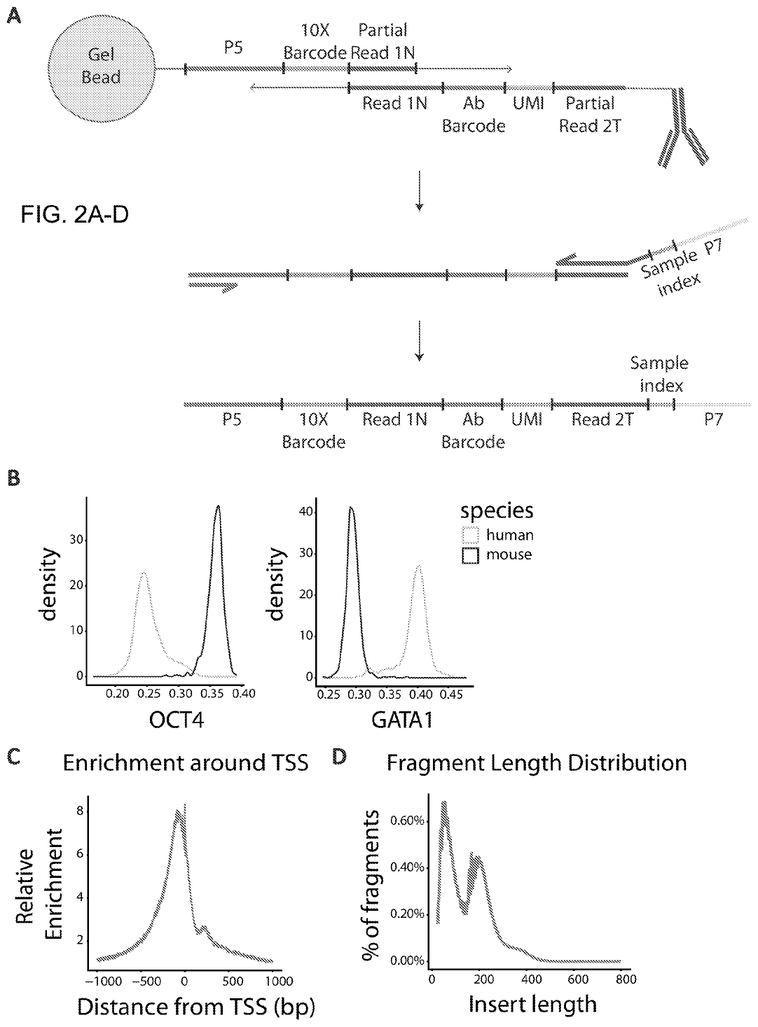

One advantage of using oligo-barcoded antibodies to measure protein levels is to enable single cell measurements of protein abundance using a sequencing read-out and to combine protein quantification with other single cell genomic assays. In an initial example, we designed antibody oligos that would be compatible with an existing kit from 10× Genomics for measuring chromatin accessibility via ATAC-seq in single cells (FIG. 2A). This allows simultaneous measurement of protein abundance along with chromatin accessibility profiles in individual cells. To test the specificity of this method for quantifying intracellular protein levels, we performed an experiment where we mixed a human erythroleukemia cell line (K562) with a mouse embryonic stem cell line (ESC) at a 1:1 ratio and stained for two transcription factors, GATA1 and OCT4, that are exclusively expressed in K562 or ESCs, respectively. We then loaded the cell mixture into the 10× Chromium controller to generate single cell emulsions and produced barcoded sequencing libraries for both the antibody-derived oligos and ATAC-seq fragments. We could then match antibody-derived oligo sequencing reads to ATAC-seq reads that originate from the same emulsion using the 10× barcode sequence that is unique to each gel bead.

To assess the specificity of the antibody-derived oligos for measuring protein abundance, we first annotated single cells as mouse ESCs or human K562 cells by determining whether their ATAC-seq fragments mapped to the mouse or human genome. We excluded cells that contain a high fraction mapping to both genomes, which represent doublets from both species, as well as potential doublets from the same species. We then quantified the fraction of unique antibody-derived oligo reads corresponding to anti-GATA1 or anti-OCT4 antibodies in ESCs vs K562s and observed specific detection of each transcription factor in their respective cell type (FIG. 2B). The ATAC-seq data generated from these cells was also of high quality despite several modifications to how the cells are prepared relative to the manufacturer's protocol, including formaldehyde fixation of the cells. The signal-to-background as measured by enrichment of ATAC-seq fragments at transcriptional start sites was well within the acceptable range for samples prepared using the regular protocol (FIG. 2C) and there was a typical distribution of fragment lengths in the ATAC-seq library (FIG. 2D). These results show that we can sensitively and specifically quantify intracellular protein levels in single cells with a sequencing read-out by using oligo-barcoded antibodies and that this method is compatible with other single cell genomic measurements.

The development of a method to measure cytoplasmic and nuclear proteins with oligo-conjugated antibodies is a valuable addition for single cell analysis. The sequence of the conjugated oligonucleotide can be designed to be compatible with any existing single cell genomics kits to combine intracellular protein measurements with these assays, as shown in FIG. 2A for the commercially available 10× Genomics single cell ATAC-seq kit. Often, key markers of specific cell types within heterogeneous tissue samples are intracellular markers and some cell states are marked by post-translational modification of specific proteins, which could not be captured with other techniques. The method described in the present examples is useful for identifying specific cells of interest and their corresponding epigenetic and/or transcriptional profiles. Furthermore, since direct epigenetic and transcriptional regulators (e.g., transcription factors) are often nuclear-localized, the ability to correlate the abundance of these regulators to chromatin state or transcriptional status will be informative for dissecting mechanisms of gene regulation and regulation of cell state.

### Example 3. Combined Assay SSB-Coated Conjugate Nuclear Protein Quantification, ATAC-Seq and RNA-Seq

This example illustrates combining nuclear protein quantification with ATAC-seq and RNA-seq within the same cell. Incubation (“staining”) is performed with oligo-conjugated antibodies as described above. In this example, when capturing and amplifying the ATAC fragments, RNA, and antibody-derived oligos, two distinct oligos can be employed as illustrated using the 10× Genomic multiomic kit protocol. Both of the gel bead oligos contain the same 10× barcode to identify the single cell from which the data originate so that the ATAC, RNA, and antibody-derived oligo reads can be traced to the same cell after sequencing.

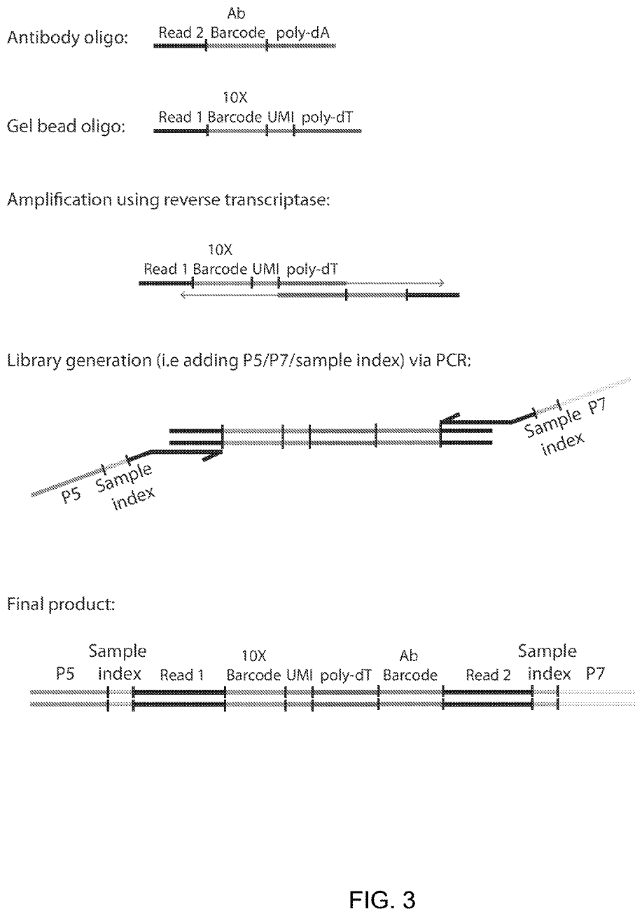

One of the oligonucleotides is used to capture and barcode ATAC fragments within the gel bead emulsion. A second poly(dT) oligonucleotide is used to capture poly(A)+mRNA, which is subsequently reverse transcribed. The poly(d)T-containing oligonucleotide can also be used to capture antibody-derived oligonucleotide if the antibody-derived oligonucleotides incorporate a poly-A tail as well. Although the antibody-derived oligo is ssDNA instead of RNA, it has been shown that reverse transcriptase can amplify using ssDNA as a template. Oligonucleotides are illustrated in FIG. 3.

Following incubation of a single cell with probes as above, sequencing libraries are generated using standard methodology. For example, the ATAC-seq libraries and RNA-seq libraries are prepared according to 10× Genomics protocols and the libraries are processed for sequencing.

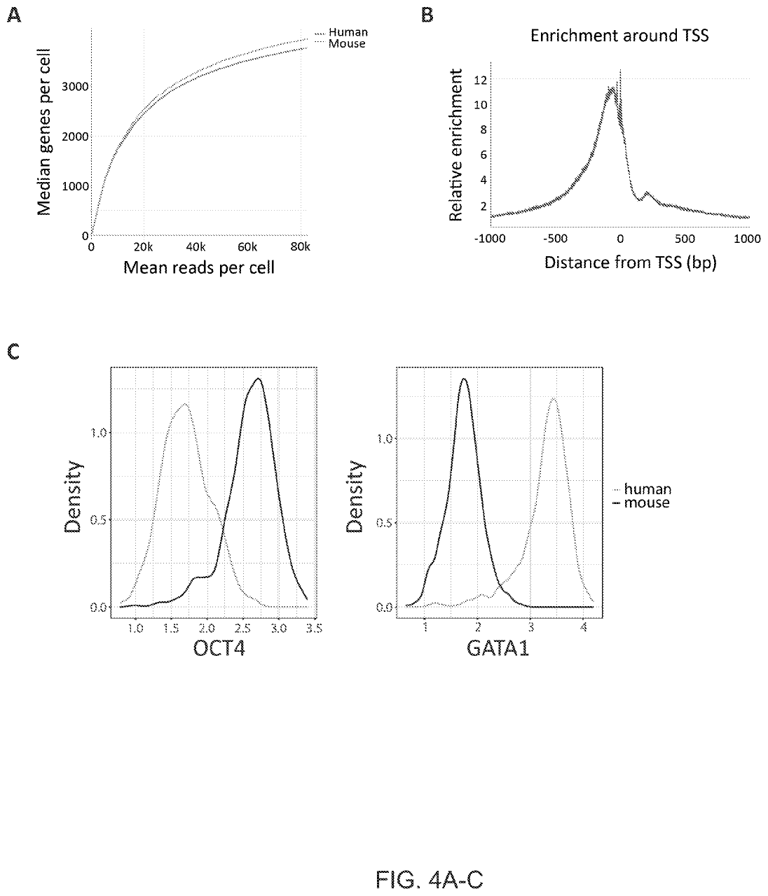

In this experiment, measurement of nuclear protein abundance using oligo-barcoded antibodies was combined with chromatin accessibility analysis and gene expression profiling in single cells. A 10× Genomics multiome kit was employed for measuring chromatin accessibility and gene expression in single cells. We amplified antibody-derived oligos using the poly-dT gel bead oligo used for capturing RNA transcripts. Antibody conjugates comprising a barcode specific for GATA1 or OCT4 transcription factors were incubated with a 1:1 mixture of human K562 and mouse ESCs. GATA1 and OCT4 transcription factors are expressed exclusively in K562 and ESCs, respectively. Single cells libraries were processed using the 10× Genomics kit. High quality data were obtained for each genomic library. The results are shown in FIG. 4A-C. FIG. 4A shows the median genes detected per cell as a function of sequencing depth as measured by mean reads per cell in the RNA-seq library. When sequencing 20,000 mean reads per cell as recommended by 10× Genomics, we detected ˜2500 genes in both human K562 and mouse ESCs which is comparable to published 10× Genomics datasets. FIG. 4B shows the TSS enrichment score for the scATAC-seq library showing high signal to noise ratios. FIG. 4C shows the distribution of the antibody-derived oligo read counts (centered log ratio normalized) for OCT4 and GATA1 in human K562 and mouse ESCs. These results demonstrated specific detection of OCT4 and GATA1 in their respective cell types.

### Example 4. Combined Protein Quantification, ATAC-Seq and RNA-Seq Using a Split-Pool Protocol

An illustrative protocol of combined protein quantification, ATAC-seq and RNA-seq using a split-pool protocol is provided below.

1. Cells are fixed and permeabilized and incubated (“stained”) with a plurality of oligonucleotide—antibody conjugates. Then, cells are treated with Tn5 transposase to obtain accessible chromatin fragments. These fragments have overhangs which will allow barcode oligonucleotides to be annealed in step 3.

2. Next, cells undergo reverse transcription using poly(dT) (or random hexamer) primers linked to a universal overhang sequence. Antibody-derived oligonucleotides can also be amplified at this step using the same oligo and reverse transcriptase.

3. Cells are washed and distributed into wells of a plate with ligation mix. Then, unique cellular indexing oligonucleotides are added to each well that will be annealed and ligated to RNA and ATAC fragments within each cell. The cellular indexing oligonucleotides have a 5′ overhang complementary to the universal overhang sequence provided by the RT primer or from the adapters inserted by Tn5 transposase, the unique cellular indexing sequence, and a 3′ overhang that will be complementary to the oligonucleotide used in the next round of indexing.

4. Cells are then pooled and redistributed for successive rounds of addition of cellular indexing sequence, with the indexing oligonucleotides containing 5′ overhangs complementary to the 3′ overhangs from the previous round of additional of indexing sequences.

5. After the last round of addition of cellular indexing sequences, cells are pooled, washed, and undergo crosslink reversal. ATAC-seq and RNA-seq library generation is completed using common protocols.

### Example 5. T Cell Profiling: Nuclear Protein Quantification in Combination with ATAC-Seq and RNA-Seq

In the present example, quantification of nuclear protein, chromatin accessibility and the transcriptome are analyzed in single cells in an illustrative assay of the present disclosure, which is referred to in this example as “NEAT-seq” (Nuclear protein Epitope Abundance, chromatin Accessibility, and the Transcriptome). Specifically, CD4 memory T cells are profiled using a panel of antibodies targeting master transcription factors (TFs) that drive T cell subsets. As described below, examples of TFs with regulatory activity gated by transcription, translation, and regulation of chromatin binding were identified. We also linked a non-coding GWAS SNP within a GATA motif to a putative target gene to internally validate GATA3-specific regulation of SNP impact. This example thus further demonstrates that antibodies to nuclear proteins comprising coated oligonucleotides can be used to measure nuclear protein abundance in single cells via sequencing, particularly in primary human samples.

We applied NEAT-seq to profile primary human CD4 memory T cells composed of distinct T cell subsets driven by known master TFs, providing a diverse system for dissecting the regulatory mechanisms upstream and downstream of these TFs to control cell state²⁰. Our antibody panel targeted TFs that drive Th1 (Tbet), Th2 (GATA3), Th17 (RORγT), and Treg (FOXP3 and Helios) cell fate²¹. After filtering, there were 8,472 cells with a median TSS enrichment of 19.0 and a median of 4,704 ATAC-seq fragments, 1,144 genes, and 1,999 RNA UMIs per cell.

We identified seven clusters in the population using scATAC-seq, which largely corresponded to clusters identified using scRNA-seq. We annotated the Th1, Th2, Th17, and Treg clusters based on master TF RNA and protein abundance, genome-wide accessibility of the TF binding motif, as well as canonical surface marker expression′. These clusters also exhibited high chromatin accessibility at functionally relevant cytokine gene loci, but low or undetectable RNA expression. This observation is suggestive of epigenetic priming of cytokine genes, where transcription is absent but the gene locus is accessible and poised for transcriptional activation, and is consistent with the primed status of memory T cells²². We also identified a small activated T cell cluster expressing activation markers CD38 and CD69²³ and a cluster with increased motif accessibility for central memory (CM) TFs, Lefl and Tcf7²⁴ and higher expression of the CM surface marker, CCR7²⁵. We annotated this cluster as CM cells, although the surrounding Th1, Th2, and Th17 clusters likely also include both CM and effector memory (EM) cells, forming a continuous “effectorness gradient” that branches out from the CM cluster into EM cells of each helper T cell subtype²⁶. Lastly, we observed a cluster lacking any distinctive markers in the scATAC-seq data, with these cells being more broadly distributed in the scRNA-seq UMAP. We hypothesized that these cells could represent uncommitted or virtual memory cells, a previously described memory cell type that arises without being stimulated by foreign antigen^{27,28}. However, it remains possible that these cells could belong to other known T cell subsets that are unidentified here.

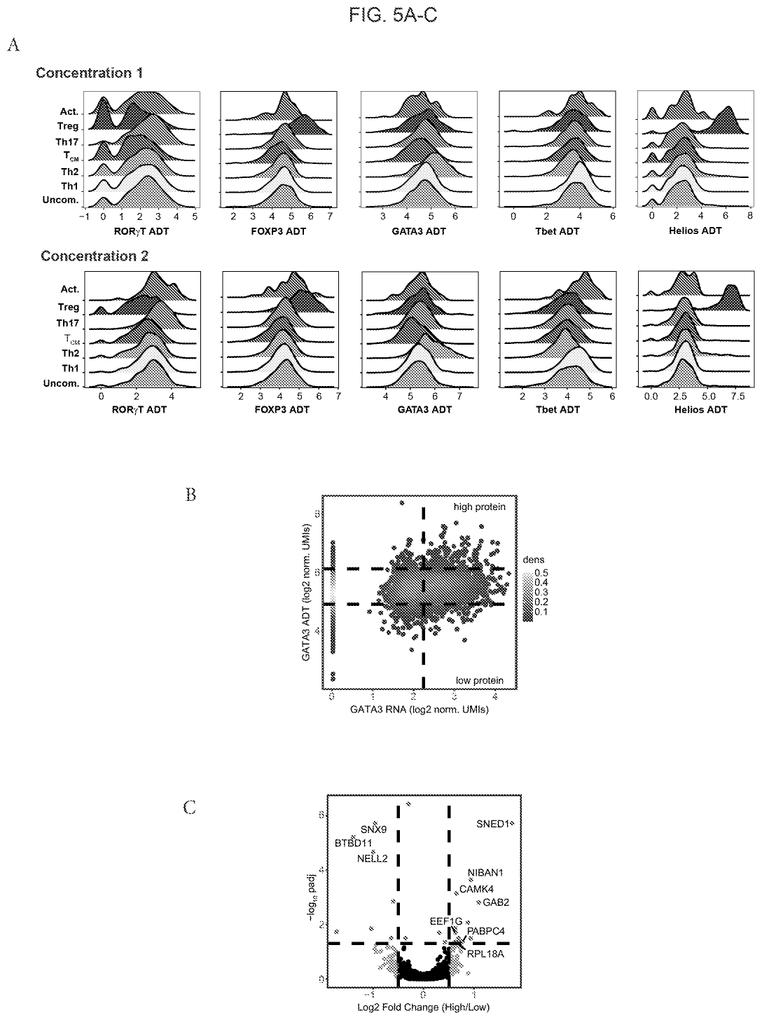

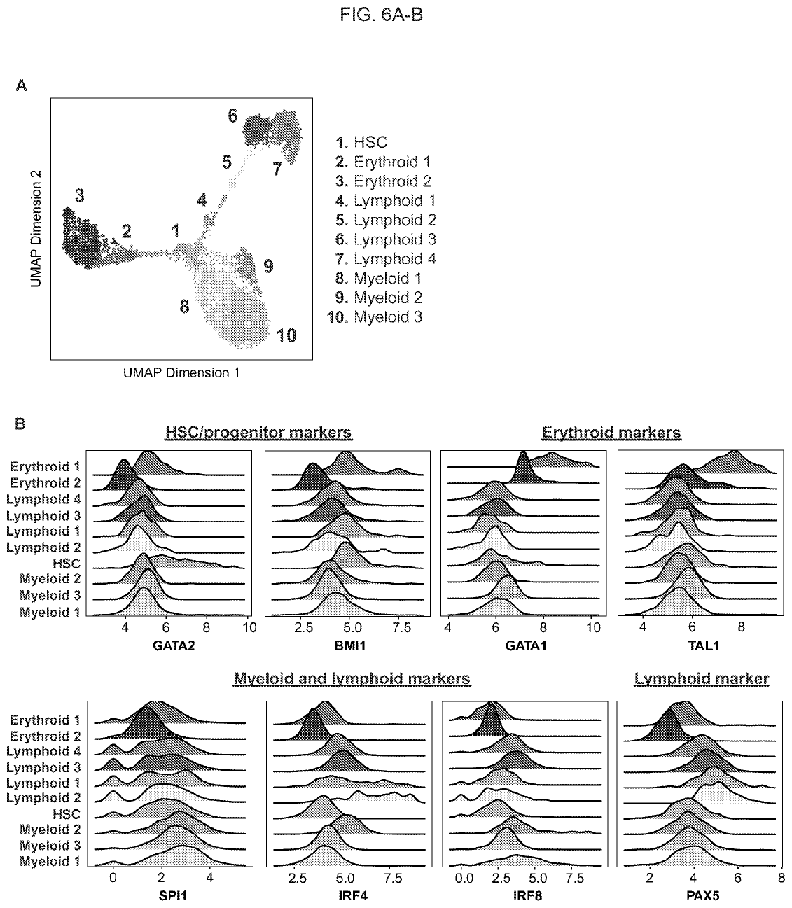

Our antibody-based measurements of protein levels for each TF showed clear enrichment in the cell type that the TF is known to drive and provided more robust detection of target TFs compared to our RNA data (FIG. 5A): smoothing of signal across neighboring cells in the UMAP was necessary for identification of cell types using RNA-seq data due to high dropout rates, while few dropouts were observed in the ADT data and unsmoothed ADT data were sufficient to clearly label cell types (FIG. 5A).

The combination of ATAC-seq and RNA-seq data with these quantitative measurements of TF protein abundance allowed us to interrogate the manner by which the expression and activity of each TF is regulated. By comparing the TF gene locus chromatin accessibility, RNA and protein abundance, and genome-wide TF binding motif accessibility across cells for each TF assayed, we identified three distinct modes of regulation in our TF panel. With RORγT and Tbet, accessibility of their gene locus was strongly correlated with the other measurements, suggesting that these TFs are regulated transcriptionally. In contrast, FOXP3 and Helios exhibited strong correlation between gene accessibility, RNA, and protein abundance but had differing patterns of motif accessibility, suggesting that their expression is regulated transcriptionally but presence of the protein does not result in chromatin remodeling. The lack of concordance between FOXP3 expression and motif accessibility is consistent with previous studies showing that FOXP3 binds to pre-existing enhancers to drive Treg fate^{√}, indicating that FOXP3 binding relies on the chromatin remodeling activity of other TFs. In the case of Helios, we believe that decoupling between protein abundance and motif activity may be due to a “collision” of binding motifs: “GGAA” is the core motif for Helios, which is highly similar to the NFAT motif, a previously described binding partner for Helios′. If Helios is mainly recruited to bind chromatin by other TFs that are expressed in a cell-type specific manner, then the Helios ChIP-seq motif, which was derived from the GM12878 B cell line, will resemble the binding motif of a recruiting TF expressed in these cells (i.e NFAT) rather than in Tregs. Supporting this hypothesis, we found that NFAT expression and NFAT motif accessibility were highly overlapping with accessibility of the B cell-derived Helios motif in CD4 memory cells, and that NFAT expression was low in Tregs. Alternatively, Helios binding may result in chromatin compaction rather than accessibility, as was recently observed in mouse hematopoietic progenitor cells′. The uncoupling of TF protein expression and motif accessibility highlight the caveats of using motif accessibility alone to infer TF activity.

The final TF in our panel, GATA3, showed clear discordance between RNA expression and protein levels across cells, with high GATA3 RNA expression observed across several memory T cell subsets and high GATA3 ADT levels observed only in the Th2 cluster. We verified specificity of our GATA3 antibody on GATA3-overexpressing cells. The ADT levels, but not RNA levels, were correlated with global changes in GATA3 motif accessibility, suggesting that ADTs faithfully report on chromatin modulating potential of this TF. These observations are consistent with post-transcriptional regulatory mechanisms restricting GATA3 protein expression in memory T cells, which could only be uncovered with the addition of protein quantification.

Our paired RNA and protein measurements also allowed us to identify candidate post-transcriptional regulators of GATA3 by performing differential expression analysis between cells expressing high levels of GATA3 RNA but low levels of protein and those expressing both high GATA3 RNA and protein (FIG. 5B). Among the top upregulated genes (FDR<0.05) were several core translation regulators, including the elongation factor EEF1G, large ribosome subunit RPL18, and poly-A binding protein PABPC4, as well as more indirect regulators such as NIBAN1, which promotes translation by regulating phosphorylation of the initiation factors EIF2A and EIF4EBP³² (FIG. 5C). GATA3 translation is regulated by PI3K signaling through mTOR 33 which, like NIBAN1, phosphorylates EIF4EBP1 to allow assembly of the initiation complex′. We also observed upregulation of a direct activator of PI3K, GAB2, in cells with high GATA3 protein levels. These results suggest that upregulation of genes that promote translation may play a role in driving GATA3 protein production in the Th2 subset of memory T cells. Together, our results identified three regulatory mechanisms used to modulate activity of the TFs in our panel: transcriptional regulation, as demonstrated by concordant RNA, protein, and motif accessibility patterns (RORγT, and Tbet); transcriptional regulation of expression but requirement of other TFs for chromatin binding (Helios and FOXP3); and translational regulation (GATA3).

In addition to using multimodal measurements to interrogate regulation of expression and activity of the TF itself, we can also use this information to uncover downstream enhancer and gene targets of a TF by correlating protein abundance of the TF with changes in regulatory element accessibility and gene expression. We found hundreds of cis-regulatory elements (i.e. ATAC-seq peaks) with accessibility significantly correlated with the protein levels of RORγT, Tbet, and GATA3 across all cells (FDR<0.05). As expected, the corresponding TF motif was significantly enriched in these peaks. We observed no significant enrichment for the FOXP3 and Helios motifs in correlated peaks, consistent with our earlier observations that these TFs are not correlated with global accessibility changes. We similarly identified dozens of genes with RNA expression significantly correlated with protein levels of each TF. Within these correlated gene sets were genes known to be enriched or functionally important in the memory T cell subset driven by the TF in question, such as IL4R for GATA3 and CTLA4 for both Helios and FOXP3³⁵.

To identify candidate genes directly regulated by each TF through a TF-associated enhancer, we overlapped the top TF ADT-correlated genes with top TF ADT-correlated scATAC-seq peaks containing the corresponding TF motif that were within 100 kb of the gene promoter and filtered for significant peak-gene linkages. We performed this analysis for the TFs that showed correlation between TF abundance and motif accessibility and identified 167 candidate TF-peak-gene linkages for GATA3, 345 for RORγT, and 81 for Tbet. These target genes were significantly enriched for GO terms related to T cell function, including T cell activation, lymphocyte differentiation, and various T cell signaling pathways. Included in these candidate TF targets were canonical surface markers for the corresponding cell type: Among the GATA3 targets were Th2 markers CCR4, CCR8, and IL4R, and among RORγT targets was the Th17 marker, CCR6.

We also reasoned that the TF-peak-gene linkages we identified could be used to interpret the effects of non-coding GWAS SNPs on TF activity and connect the SNPs to putative target genes. We overlapped peaks in our TF-peak-gene linkages with candidate causal GWAS SNPs 36 and identified a SNP, rs62088464, located within a GATA motif sequence in a GATA3 ADT-associated peak. The risk allele, which preserves the GATA motif, is associated with decreased pulmonary function as measured by decreased forced vital capacity³⁷, which can result from pulmonary fibrosis and other inflammatory lung diseases associated with Th2 immune responses³⁸. The gene linked to the peak containing this SNP encodes the tRNA splicing endonuclease, TSEN54, a gene with significantly enriched expression in the sputum of patients with type-2 airway inflammation 39,4°. Since our T cell donor was heterozygous for this SNP, we examined whether the risk allele was more accessible than the protective allele in cells with high GATA3 protein levels. Indeed, we observed that almost all ATAC-seq reads in the top 10% of cells ranked by GATA3 ADT levels mapped to the risk allele, while this difference was far less pronounced in cells with lower levels of GATA3 ADT (p=7.81λ10⁻⁴). Similarly, the risk allele is associated with increased TSEN54 expression in GTEx data and TSEN54 was the gene most strongly associated with the risk allele in various tissues. Together, these results suggest that GATA3 binds the risk allele sequence to activate the regulatory element and drive expression of TSEN54 and that this binding is disrupted with the protective allele.

This example thus demonstrated that NEAT-seq provides a robust method for studying the quantitative effects of epigenetic regulator abundance on both chromatin and gene expression state in primary human samples. Whereas previous studies investigating dosage-dependent effects of TFs often required building cell lines with a combination of hypomorphic and null alleles^{41,42} or inducible expression systems⁴³, we demonstrated that our technique can measure the molecular consequences of continuous changes in TF levels in a biologically relevant setting for a panel of proteins simultaneously. Since nuclear proteins encompass many proteins involved in gene regulation including TFs and chromatin modifiers, the capacity to link nuclear protein levels to epigenetic and transcriptional status provides a powerful approach for studying gene regulation. While oligo-antibodies against nuclear proteins are currently limited, we anticipate that these will become more readily available as demand increases. Incorporating additional modalities such as cytoplasmic and cell surface proteins, CRISPR gRNA sequencing, and TCR sequencing will enable measurement of the effects of cellular perturbations and signaling pathways on cell state, providing an even more comprehensive picture of cellular programs.

### Example 6. Primary Human Bone Marrow Nuclear Protein Quantification in Combination with ATAC-Seq and RNA-Seq

We stained primary human bone marrow mononuclear cells (BMIVICs) with oligo antibodies targeting eight nuclear proteins (7 transcription factors and 1 chromatin remodeler) and performed NEAT-seq (i.e., profiled ATAC-seq, RNA-seq, and levels of the targeted nuclear proteins in single cells using the 10× Genomics Multiome kit). The nuclear proteins targeted are the nuclear protein markers were enriched in the expected cell types relative to other cell types in the population. The experiments were conducted using the methodology described for Example 5.

### Methods—Example 5

### Cell Culture

Frozen vials of primary human CD4+CD45R0+ memory T cells were purchased from STEMCELL Technologies (Cat #70031).

### Antibody Conjugation

The nuclear pore complex antibody (Biolegend 902901) was conjugated with streptavidin using the Lightning-Link Streptavidin Conjugation Kit from Abcam (ab102921) according to manufacturer's instructions. NaCl and Tween were added to the conjugated antibody mixture to a final concentration of 0.5M NaCl and 0.01% Tween and mixed with biotinylated oligos (purchased from IDT) at equimolar ratio. The mixture was incubated overnight at room temperature and unbound oligo was removed using Amicon 100KDa centrifugal filters (UFC510008). Antibody conjugates were eluted and stored in PBS. Antibodies in the TF panel for CD4 memory T cells were directly conjugated to oligos by BD Biosciences. The antibodies in the panel were the following clones from BD Biosciences: GATA3 (L50-823), Tbet (4B10), RORγT (Q21-559), FOXP3 (259D/C7), and Helios (22F6).

### Binding of Single Stranded DNA Binding Protein to Oligo-Antibodies

To bind EcoSSB (Promega M3011) to the antibody-oligos, we incubated the antibody and EcoSSB in 50u1 of 1× NEBuffer 4 for 30 min at 37 degrees Celsius. We then added a final concentration of 3% BSA, 1× PBS, and 1U/ul RNase inhibitor directly to the antibody-EcoSSB mix (without any purification) in a final volume of 100u1 for staining cells. To calculate the amount of EcoSSB needed to saturate binding sites on the antibody oligos, we estimated that each antibody was conjugated to an average of 2 oligos of 95 bp, and each EcoSSB tetramer would bind with a ˜35 bp footprint^{44,45}, requiring 6 EcoSSB tetramers per antibody. Based on the concentration of antibody being used and reported K^{d} of EcoSSB (in the ˜2 nM range)¹⁷, we can then estimate the amount of EcoSSB necessary to bind a given fraction of oligos (aiming for >0.9) using the following equation:

$$desired fraction of oligo bound = \frac{\begin{matrix}( {[ E c o S S B ]}_{t o t} + {[ oligo ]}_{t o t} + {K}_{d} ) - \\ \sqrt{\begin{matrix}{( {[ E c o S S B ]}_{t o t} + {[ oligo ]}_{t o t} + {K}_{d} )}^{2} - \\ 4 * {[ E c o S S B ]}_{t o t} * {[ oligo ]}_{t o t}\end{matrix}}\end{matrix}}{2 * {[ oligo ]}_{t o t}}$$

$${where [ oligo ]}_{t o t} = antibody concentration * 2 oligos * 3 EcoSSB binding sites per oligo .$$

### Oligo-Antibody Staining

Cells were fixed in 1.6% formaldehyde in PBS for 2 min at room temperature, then quenched with 0.25M glycine for 5 min on ice and spun down at 600g for 5 min. Cells were washed twice with PBS and then resuspended in lysis/permeabilization buffer (20 mM Tris-HCl pH 7.5, 150 mM NaCl, 3 mM MgCl2, 0.5% NP40, 0.1% Tween-20, 0.01% digitonin, 1U/ul RNase inhibitor, 1 mM DTT). Cells were incubated on ice for 10 mins, pelleted at 600g for 5 mins, and washed twice with wash buffer (20 mM Tris-HCl pH 7.5, 150 mM NaCl, 3 mM MgCl2, 0.1% Tween-20, 1U/ul RNase inhibitor, 1 mM DTT). Cells were incubated in staining buffer (PBS with 3% BSA, 1U/ul RNase inhibitor) with 1 mM DTT and 1 mg/ml of single stranded DNA (ssDNA) for 30 mins at room temperature, pipetting often to resuspend cells. For the flow cytometry experiments involving GFP staining, salmon sperm DNA was used for the ssDNA block. However, due to significant amounts of annealing to form double stranded DNA that would result in contaminating reads in ATAC-seq data, we switched to using either a mixture of random 30-mers or a 30 bp ssDNA oligo sequence with no complementarity to the mouse or human genome for multiome experiments. To ensure no priming would occur with these oligos, they were modified with a terminal dideoxy cytosine.

After blocking with ssDNA, Tween was added to a final concentration of 0.1% and cells were pelleted and washed once with staining buffer+0.1% Tween. Cells were then split into 5 tubes and each tube of cells was incubated with an anti-NPC antibody linked to a distinct HTO (pre-bound with SSB) for 30 min at room temperature. Cells were washed twice with staining buffer+0.1% Tween, re-pooled, and incubated with TF antibody mix for 30 min at room temperature. For the CD4 memory T cell experiment, cells were split into two tubes prior to incubating with two concentrations of the TF antibody mix. A distinct hashing antibody was also added to the two TF antibody mixes to identify the concentration of antibody that each cell was stained with. Cells were then washed twice with staining buffer+0.1% Tween, and cells incubated with different concentrations of TF antibody were pooled. Cells were washed once more with PBS containing 1% BSA and 1U/u1 RNase inhibitor, then resuspended in 1× Nuclei buffer containing 1U/u1 RNase inhibitor from the 10× Genomics Multiome kit. The cell suspension was then filtered through a 40 um Flowmi strainer 2-3 times until nuclei clusters were removed.

inCITE-seq staining conditions were performed as described in Chung et al. 2021. For NEAT-seq fixation and permeabilization followed by staining using inCITE-seq staining conditions, we performed fixation and permeabilization as described above and then proceeded with the dextran sulfate blocking and staining conditions (1:100 FcX (BioLegend 156604)+1% BSA+0.05% Dextran Sulfate) employed by inCITE-seq.

### Antibody Concentrations

The NPC antibodies were used at 0.3 μg in 100 11.1 of staining buffer (3 ug/mL). The two antibody concentrations for TF antibodies used in the CD4 memory T cell experiment are indicated below:

| | Conc. 1 (ug/mL; marked by | Conc. 2 (ug/mL; marked by |
|---|---|---|
| Antibody | NPC1) | NPC2) |
| RORγT | 0.39075 | 1.95375 |
| Foxp3 | 2.5 | 5 |
| GATA3 | 3.125 | 15.625 |
| Helios | 0.39075 | 1.95375 |
| Tbet | 3.125 | 15.625 |

Both antibody concentrations showed specific staining of the targeted TF in the appropriate cell type, as shown in FIG. 5A. We chose concentration 2 for follow-up analyses since it provided slightly better enrichment over background for some antibodies.

### Single Cell Library Preparation and Sequencing

Antibody-stained cells in 1× Nuclei buffer were processed using the 10× Genomics Multiome kit as indicated in the standard protocol (Rev A) to generate ATAC-seq and RNA-seq libraries. For the CD4 memory T cell experiment, 6,000 cells were targeted per lane and 2 lanes were used. During the pre-amplification step, Truseq read 2 (CAGACGTGTGCTCTTCCGATC) and Nextera read 2 (GGCTCGGAGATGTGTATAAGAGACAG) primers were spiked in at 0.2 uM final concentration to amplify ADT and HTO oligos. To generate ADT and HTO libraries, 35u1 of pre-amplification product from step 4.3p was amplified with indexing primers using 2× NEB Next High-Fidelity PCR Master Mix (M0541). A double-sided SPRI bead clean up was performed using 0.6× SPRI beads (retaining supernatant) and then adding additional SPRI beads to a final concentration of 1.2×, washing with 80% ethanol, and eluting ADT or HTO libraries from beads using EB buffer. Libraries were quantified by PCR using a PhiX control v3 (Illumina FC-110-3001) standard curve. scATAC-seq libraries were sequenced alone on a NextSeq 550 sequencer and ADT libraries were sequenced together with scRNA-seq libraries on a NextSeq 550. Recommended sequencing read configurations for 10× Multiome libraries were used for scATAC- and scRNA-seq libraries. We sequenced approximately 40,000 read pairs per cell for scATAC-seq, 35,000 read pairs per cell for scRNA-seq libraries, and 5,000 read pairs per cell for both the ADT and HTO libraries in the CD4 memory T cell experiment.

### Antibody Oligo Sequences

ADT oligos had a partial Truseq read 2 sequence followed by 12 bp UMI, 36 bp antibody-specific barcode, and 25 bp poly A tail as follows:

CAGACGTGTGCTCTTCCGATCT[12 bp UMI]
[36 bp Barcode]AAAAAAAAAAAAAAAAAA
AAAAAAA

HTOs were similarly designed, except they instead had a partial Nextera read 2 sequence to allow separate amplification of TF antibody oligos from HTOs, which often stain at higher levels:

GGCTCGGAGATGTGTATAAGAGACAG[12 bp UMI]
[36 bp Barcode]AAAAAAAAAAAAAAAAAAAAAA
AAA

Note that the hashing antibody used together with the TF antibody panel for marking the two antibody concentrations tested in CD4 memory T cells was linked to an ADT oligo with a partial Truseq read 2 sequence so that it would be amplified with the TF ADTs and could be used to normalize TF ADT counts.

### Analytical Methods

### ADT and HTO Processing

Raw sequencing data were converted to fastq format using bcl2fastq (Illumina). ADTs and HTOs were then assigned to individual cells and antibodies using the matcha barcode matching tool′. Cell barcodes were matched based on exact matches, and up to 3 mismatches were allowed in antibody barcode sequences. Counts for each antibody were tabulated by counting UMIs. Cells with fewer than 75 HTO UMIs or 100 ADT UMIs were excluded. TF ADT counts were normalized to HTO counts from the anti-NPC HTO that was added to distinguish two different concentrations of the TF antibody panel used to stain cells, since we expected that levels of the nuclear pore complex should be relatively constant across cells. We observed very similar results when normalizing to total ADT counts or just using raw ADT counts. We then multiplied by 250 (i.e roughly the median number of NPC counts per cell), added one pseudocount, and loge-transformed counts. We chose the NPC normalization method because it was more robust than centered log ration (CLR) transformation in cases where cells are primarily positive for only one antibody in the panel, as was the case for the CD4 memory T cells.

### Doublet Detection Using HTOs

For doublet detection in the CD4 memory T cell experiment, we filtered for cells with at least 75 HTO counts per cell and performed CLR-transformation on HTO counts only. We set CLR cutoffs for positive staining of each HTO individually based on the bimodal distribution for each HTO and only cells positive for exactly one HTO were retained. Since we also incorporated two hashing oligos in the TF staining step to distinguish between two antibody concentrations used, we also annotated doublets using these HTOs and removed them from analysis.

scATAC-Seq Analysis

Raw sequencing data were converted to fastq format and aligned to the hg38 reference genome using cellranger-ARC v.1.0.1 from 10× Genomics. Fragment files were then loaded into ArchR (v1.0.2) using the createArrowFiles function. Cells with a TSS enrichment<10 or fewer than 1000 unique fragments per cell were removed from analysis along with HTO-annotated doublets. Remaining cells were projected onto a reference dataset of hematopoietic cells⁴⁸, using a liftover of the published hg19 peak coordinates to hg38 and the published LSI loadings for each peak. Cell type annotations were transferred as the most common cell type from the 10 nearest neighbors, and contaminating CD8 memory T cells were removed from further analysis. We next computed an iterative LSI dimensionality reduction using the addIterativeLSI function with the default tile matrix (insertion counts in 500 bp bins across the genome) and 4 iterations. Clustering was then performed using the addClusters function and a UMAP was generated using addUMAP, both with default parameters.

To call peaks, we first generated insertion coverage files from pseudobulk replicates grouped by cluster using addGroupCoverages and then called peaks with macs2 using addReproduciblePeakSet with default parameters. We then generated a matrix of insertion counts for each peak across all cells using addPeakMatrix. To aid in cluster identification, we identified marker peaks unique to each cluster and identified TF motifs enriched in these peaks using getMarkerFeatures (useMatrix=“PeakMatrix”) and peakAnnoEnrichment. Results were plotted using plotEnrichHeatmap(enrichMotifs, n=5, transpose=TRUE, cutOff=5). We can also predict TF activity by measuring differences in TF motif accessibility across cells using chromVAR⁴⁹. We first determined which peaks contain a motif of interest for motifs in the CISBP database 5° using addMotifAnnotations with the option motifSet=“cisbp”. We then added a background peak set with similar GC content and number of fragments and computed motif deviations for all motifs using addBgdPeaks and addDeviationsMatrix, respectively.

To further help with cluster identification using ATAC-seq data, we can predict gene expression or epigenetic priming of a locus by calculating gene activity scores for each gene based on accessibility in the region surrounding the gene locus. These scores were calculated in ArchR during Arrow file creation with the option addGeneScoreMat=TRUE.

scRNA-Seq Analysis

Raw sequencing data were converted to fastq format and aligned to the reference genome using cellranger-ARC v.1.0.1 from 10× Genomics. For each lane, the gene expression matrix from the filtered_feature_bc_matrix was used to create a Seurat object using Seurat v3.2.1. The two lanes of CD4 memory T cell data were then merged into one Seurat object and filtered for cells used in the scATAC-seq analysis. Data were normalized with NormalizeData (normalization.method=“LogNormalize” and scale.factor=10000). For principal component analysis, we identified the top 2000 variable genes using FindVariableFeatures (selection.method=“vst”) and RunPCA was performed on scaled data using these variable features. We then clustered cells using FindNeighbors with dimensions 1:15 and FindClusters with resolution 0.6. The RNA UMAP was generated with RunUMAP using dimensions 1:15. FindAllMarkers was used to identify marker genes enriched in each cluster.

To identify candidate regulators of GATA3 translation, we added ADT data to our Seurat object using CreateAssayObject. We first filtered for cells expressing high GATA3 RNA (natural log-normalized counts>2.25) and then identified cells expressing high GATA3 ADT (log 2 NPC-normalized counts>6.12) or low GATA3 ADT (log 2 NPC-normalized counts<4.9116 to match number of cells in high GATA3 ADT subset). To identify differentially expressed genes between these two subsets, we ran FindMarkers. We converted the natural log-based fold change values output from Seurat v3 to log 2 fold changes and calculated adjusted p values using Benjamini-Hochberg correction.

### Data Visualization

Unless otherwise indicated in the text, visualization of TF motif deviation Z-scores, gene activity scores, RNA, and ADTs on the ATAC UMAP embedding was done by plotting imputed values using ArchR's plotEmbedding function. Ridge plots of normalized ADT counts and scatterplots with marginal histograms of normalized ADT vs RNA counts were generated using ArchR's plotGroups (plotAs=“ridges”) and ggpubr's ggscatterhist, respectively. Normalized ADT counts were calculated as log 2(250*(TF ADT counts/NPC HTO counts)+1). Normalized RNA counts were calculated as log 2(10000*(TF RNA counts/total UMI counts)+1).

Identifying Peaks and Genes Correlated with TF Abundance

To identify peaks and genes with changes that correlate with TF ADT levels, Spearman correlation values were calculated between normalized ADT counts for each TF and either normalized Tn5 insertion counts or normalized RNA counts for all peaks and genes with >10 observed reads across single cells. Raw p-values for correlations were calculated in the same manner as R cor.test, namely using a two-sided t-test with n-2 degrees of freedom where

$$t = ρ \sqrt{\frac{n - 2}{1 - {ρ}^{2}}}$$

and n is the number of cells. P-values were multiple-hypothesis corrected for each ADT using the “BH” method of R's p.adjust, and significant correlations were defined as adjusted p-value<0.05. TF motif enrichment in significantly correlated peaks was calculated using a hypergeometric test.

### Identification of Correlated Peaks and Genes

To identify peaks and genes where peak accessibility correlated with gene expression, we formed 500 aggregates of 100 cells each using the 99 nearest neighbors of randomly selected cells in LSI coordinates. These aggregates were constrained to have a maximum pairwise overlap of 80% of cells. Gene expression and peak accessibility for each aggregate was calculated by averaging the normalized accessibility or expression values across all cells in the aggregate. For all peak-gene pairs within 100 kb of each other, we calculated Spearman correlation and significance using a two-sided t-test as for our peak-TF and gene-TF correlations.

### Identifying TF-Peak-Gene Linkages

To identify candidate direct target genes of a TF, we identified TF ADT-correlated genes that had a TF ADT-correlated peak nearby containing the TF sequence motif. Specifically, we overlapped the top 20% of ADT-correlated genes with the top 20% of ADT-correlated peaks containing the corresponding TF motif, sorted by Spearman correlation calculated across single cells. For the overlap, we required that the peak-gene distance be less than 100 kb and that accessibility of the peak and expression of the linked gene be significantly correlated (adjusted p-value<0.05 for Spearman correlation, as described above). To identify GO terms enriched in these genes, we used the enrichGO function in the clusterProfiler R package⁵², using all genes with at least 1 RNA count across all cells in our dataset as the background gene list.

### Analysis of Fine-Mapped GWAS Variants

To identify candidate causal SNPs regulated by a TF and link the SNP to a putative target gene, we obtained a comprehensive list of fine-mapped GWAS SNPs (see https site pics2.ucsf.edu/PICS2.html) and overlapped these with peaks from our identified GATA3 TF-peak-gene linkages. We focused on rs62088464, a SNP located within a GATA motif site and for which our donor was heterozygous for the risk allele. To determine allele-specific differences in accessibility at this SNP, we identified all reads overlapping this SNP with mapq>30 using pysam's pileup method^{59,60}. To stratify cells by GATA3 expression, we z-score transformed the CLR-normalized GATA3 expression levels for each of the two antibody titration levels to ensure they were on comparable scales, then performed smoothing using the ArchR version of the MAGIC algorithm to reduce noise. Cells were divided based on their rank in the smoothed GATA3 vector. Allele-specific accessibility was determined using a one-sided binomial test, comparing the allele frequency in the top 10% of GATA3 cells using the bottom 50% as a null hypothesis. The eQTL data and analysis shown were obtained from the GTEx Portal release v8.

### REFERENCES CITED BY NUMBER IN THE EXAMPLES SECTION

1. Ma, S. et al. Chromatin Potential Identified by Shared Single-Cell Profiling of RNA and Chromatin. Cell 183, 1103-1116.e20 (2020).
2. Stoeckius, M. et al. Simultaneous epitope and transcriptome measurement in single cells. Nat. Methods 14, 865-868 (2017).
3. Swanson, E. et al. Simultaneous trimodal single-cell measurement of transcripts, epitopes, and chromatin accessibility using TEA-seq. Elife 10, (2021).
4. Mimitou, E. P. et al. Scalable, multimodal profiling of chromatin accessibility, gene expression and protein levels in single cells. Nat. Biotechnol. (2021) doi:10.1038/s41587-021-00927-2.
5. Chung, H. et al. Simultaneous single cell measurements of intranuclear proteins and gene expression. doi:10.1101/2021.01.18.427139.
6. Gerlach, J. P. et al. Combined quantification of intracellular (phospho-)proteins and transcriptomics from fixed single cells. Sci. Rep. 9, 1469 (2019).
7. Reimegard, J. et al. A combined approach for single-cell mRNA and intracellular protein expression analysis. Commun Biol 4, 624 (2021).
8. Rivello, F. et al. Single-cell intracellular epitope and transcript detection revealing signal transduction dynamics. doi:10.1101/2020.12.02.408120.
9. Spitz, F. & Furlong, E. E. M. Transcription factors: from enhancer binding to developmental control. Nat. Rev. Genet. 13, 613-626 (2012).
10. Gran, D., Kester, L. & van Oudenaarden, A. Validation of noise models for single-cell transcriptomics. Nat. Methods 11, 637-640 (2014).
11. Marinov, G. K. et al. From single-cell to cell-pool transcriptomes: stochasticity in gene expression and RNA splicing. Genome Res. 24, 496-510 (2014).
12. Gillespie, M. A. et al. Absolute Quantification of Transcription Factors Reveals Principles of Gene Regulation in Erythropoiesis. Mol. Cell 78, 960-974.ell (2020).
13. Mimitou, E. P. et al. Multiplexed detection of proteins, transcriptomes, clonotypes and CRISPR perturbations in single cells. Nat. Methods 16, 409-412 (2019).
14. Stoeckius, M. et al. Cell Hashing with barcoded antibodies enables multiplexing and doublet detection for single cell genomics. Genome Biol. 19, 224 (2018).
15. Wang, Y. et al. Multiplexed in situ protein imaging using DNA-barcoded antibodies with extended hybridization chain reactions. doi:10.1101/274456.
16. Molineux, I. J., Pauli, A. & Gefter, M. L. Physical studies of the interaction between the Escherichia coli DNA binding protein and nucleic acids. Nucleic Acids Res. 2, 1821-1837 (1975).
17. Reddy, M. S., Guhan, N. & Muniyappa, K. Characterization of single-stranded DNA-binding proteins from Mycobacteria. The carboxyl-terminal of domain of SSB is essential for stable association with its cognate RecA protein. J. Biol. Chem. 276, 45959-45968 (2001).
18. Marceau, A. H. Functions of single-strand DNA-binding proteins in DNA replication, recombination, and repair. Methods Mol. Biol. 922, 1-21 (2012).
19. Chung, H. et al. Joint single-cell measurements of nuclear proteins and RNA in vivo. Nat. Methods 18, 1204-1212 (2021).
20. Sallusto, F. & Lanzavecchia, A. Heterogeneity of CD4+ memory T cells: functional modules for tailored immunity. Eur. J. Immunol. 39, 2076-2082 (2009).
21. Fang, D. & Zhu, J. Dynamic balance between master transcription factors determines the fates and functions of CD4 T cell and innate lymphoid cell subsets. J. Exp. Med. 214, 1861-1876 (2017).
22. Barski, A. et al. Rapid Recall Ability of Memory T cells is Encoded in their Epigenome. Sci. Rep. 7, 39785 (2017).
23. Motamedi, M., ×u, L. & Elahi, S. Correlation of transferrin receptor (CD71) with Ki67 expression on stimulated human and mouse T cells: The kinetics of expression of T cell activation markers. Journal of Immunological Methods vol. 437 43-52 (2016).
24. Durek, P. et al. Epigenomic Profiling of Human CD4 T Cells Supports a Linear Differentiation Model and Highlights Molecular Regulators of Memory Development. Immunity 45, 1148-1161 (2016).
25. Sallusto, F., Lenig, D., Förster, R., Lipp, M. & Lanzavecchia, A. Two subsets of memory T lymphocytes with distinct homing potentials and effector functions. Nature 401, 708-712 (1999).
26. Cano-Gamez, E. et al. Single-cell transcriptomics identifies an effectorness gradient shaping the response of CD4 T cells to cytokines. Nat. Commun. 11, 1801 (2020).
27. CD4+ virtual memory: Antigen-inexperienced T cells reside in the naïve, regulatory, and memory T cell compartments at similar frequencies, implications for autoimmunity. J. Autoimmun. 77, 76-88 (2017).
28. Kawabe, T. et al. Memory-phenotype CD4 T cells spontaneously generated under steady-state conditions exert innate Tl-like effector function. Sci Immunol 2, (2017).
29. Samstein, R. M. et al. Foxp3 exploits a pre-existent enhancer landscape for regulatory T cell lineage specification. Cell 151, 153-166 (2012).
30. Gabriel, C. H. et al. Identification of Novel Nuclear Factor of Activated T Cell (NFAT)-associated Proteins in T Cells. J. Biol. Chem. 291, 24172-24187 (2016).
31. Cova, G. et al. Helios represses megakaryocyte priming in hematopoietic stem and progenitor cells. J. Exp. Med. 218, (2021).
32. Sun, G. D. et al. The endoplasmic reticulum stress-inducible protein Niban regulates eIF2alpha and S6K1/4E-BP1 phosphorylation. Biochem. Biophys. Res. Commun. 360, 181-187 (2007).
33. Cook, K. D. & Miller, J. TCR-dependent translational control of GATA-3 enhances Th2 differentiation. J. Immunol. 185, 3209-3216 (2010).
34. Regulation of Translation Initiation in Eukaryotes: Mechanisms and Biological Targets. Cell 136, 731-745 (2009).
35. Schmiedel, B. J. et al. Impact of Genetic Polymorphisms on Human Immune Cell Gene Expression. Cell 175, 1701-1715.e16 (2018).
36. Taylor, K. E., Mark Ansel, K., Marson, A., Criswell, L. A. & Farh, K. K.-H. PICS2: next-generation fine mapping via probabilistic identification of causal SNPs. Bioinformatics (2021) doi:10.1093/bioinformatics/btab122.
37. Alkes Group. https://alkesgroup.broadinstitute.org/.
38. Gieseck, R. L., Wilson, M. S. & Wynn, T. A. Type 2 immunity in tissue repair and fibrosis. Nature Reviews Immunology vol. 18 62-76 (2018).
39. Peters, M. C. et al. A Transcriptomic Method to Determine Airway Immune Dysfunction in T2-High and T2-Low Asthma. Am. J. Respir. Crit. Care Med. 199, 465-477 (2019).
40. Singh, D. et al. COPD patients with chronic bronchitis and higher sputum eosinophil counts show increased type-2 and PDE4 gene expression in sputum. J. Cell. Mol. Med. 25, 905-918 (2021).
41. Affar, E. B. et al. Essential dosage-dependent functions of the transcription factor yin yang 1 in late embryonic development and cell cycle progression. Mol. Cell. Biol. 26, 3565-3581 (2006).
42. Takeuchi, J. K. et al. Chromatin remodelling complex dosage modulates transcription factor function in heart development. Nat. Commun. 2, 187 (2011).
43. Sokolik, C. et al. Transcription factor competition allows embryonic stem cells to distinguish authentic signals from noise. Cell Syst 1, 117-129 (2015).
44. Bujalowski, W. & Lohman, T. M. Escherichia coli single-strand binding protein forms multiple, distinct complexes with single-stranded DNA. Biochemistry 25, 7799-7802 (1986).
45. Lohman, T. M. & Overman, L. B. Two binding modes in Escherichia coli single strand binding protein-single stranded DNA complexes. Modulation by NaCl concentration. J. Biol. Chem. 260, 3594-3603 (1985).
46. Benjamin Parks. GreenleafLab/matcha. https://github.com/GreenleafLab/matcha.
47. Stuart, T. et al. Comprehensive Integration of Single-Cell Data. Cell 177, 1888-1902.e21 (2019).
48. Granja, J. M. et al. Single-cell multiomic analysis identifies regulatory programs in mixed-phenotype acute leukemia. Nat. Biotechnol. 37, 1458-1465 (2019).
49. Schep, A. N., Wu, B., Buenrostro, J. D. & Greenleaf, W. J. chromVAR: inferring transcription-factor-associated accessibility from single-cell epigenomic data. Nat. Methods 14, 975-978 (2017).
50. Weirauch, M. T. et al. Determination and inference of eukaryotic transcription factor sequence specificity. Cell 158,1431-1443 (2014).
51. van Dijk, D. et al. Recovering Gene Interactions from Single-Cell Data Using Data Diffusion. Cell 174, 716-729.e27 (2018).
52. Yu, G., Wang, L.-G., Han, Y. & He, Q.-Y. clusterProfiler: an R Package for Comparing Biological Themes Among Gene Clusters. OMICS: A Journal of Integrative Biology vol. 16 284-287 (2012).

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference with respect to the material for which they are expressly cited.

1. A method of quantifying the levels of a plurality of cellular proteins present in a cell, the method comprising
incubating a cell with a population of binding moiety-oligonucleotide conjugates comprising a plurality of conjugates in which each conjugate comprises a target binding moiety that specifically binds to a cellular protein to be quantified conjugated to an oligonucleotide that comprises a target-binding-moiety barcode sequence, said target-binding-moiety barcode sequence differing in sequence from target-binding-moiety barcode sequences contained in oligonucleotide components of conjugates that comprise different target binding moieties,
wherein each of the plurality of conjugates comprises a nucleic acid binding protein bound to each oligonucleotide component; and
quantifying the level of barcode sequences for each binding moiety associated with the cell, thereby quantifying the level of each of the cellular proteins bound to the target binding moiety.

2. The method of claim 1, further comprising a step of performing an amplification reaction to amplify oligonucleotide sequences of conjugates bound to cellular proteins to obtain an amplification product.

3. The method of claim 2, comprising incorporating a cellular identification sequence, a unique molecular identifier (UMI) sequence, and/or a sample identification sequence during amplification.

4. The method of claim 2, wherein quantifying the level of barcode sequences comprises a quantitative amplification reaction; and or massively parallel sequencing.

5-6. (canceled)

7. The method of claim 1, wherein the oligonucleotide comprises a fluorescent label; or the oligonucleotide hybridizes to a complementary oligonucleotide that comprises a fluorescent label.

8. The method of claim 7, further comprising detecting a signal from the fluorescent label to localize the position of the target binding moiety in the cell and/or quantifying the signal from the fluorescent label.

9. (canceled)

10. The method of claim 1, wherein the nucleic acid binding protein is a sequence non-specific nucleic acid binding protein.

11. The method of claim 10, wherein the nucleic acid binding protein preferentially binds to single stranded DNA.

12. The method of claim 1, wherein the nucleic acid binding protein is Escherichia coli SSB or T4 gene 32 protein.

13. The method of claim 1, wherein the cell is a permeabilized cell.

14. The method of claim 1, wherein the binding moiety comprises an antibody, or a binding fragment thereof, that specifically binds to the target cellular protein; a ligand that binds to a site on the target cellular protein; or an aptamer that specifically binds to the target cellular protein; and/or one or more target polypeptides is an intracellular protein.

15-19. (canceled)

20. The method of claim 1, wherein incubation comprises incubating a plurality of cells with the population of binding moiety-oligonucleotide conjugates; optionally wherein the plurality of cells is present in a tissue sample.

21. The method of claim 20, further comprising
compartmentalizing single cells of the population into single cell analysis compartments.

22. The method of claim 20, wherein compartmentalizing is performed after incubating the plurality of cells with the population of binding moiety-oligonucleotide conjugates; and/or the method further comprises at least one washing step following incubation prior to compartmentalization.

23-25. (canceled)

26. The method of claim 20, wherein the plurality of cells is present in a tissue sample and wherein the tissue sample is a section of a tissue and the oligonucleotide further comprises a region that specifically hybridizes to a complementary oligonucleotide that comprises a positional barcode sequence that is specific for a position in the tissue.

27. A method of quantifying the levels of a plurality of cellular proteins present in a cell, the method comprising
(a) incubating a plurality of cells with a population of binding moiety-oligonucleotide conjugates comprising a plurality of conjugates in which each conjugate comprises a target binding moiety that specifically binds to a cellular protein to be quantified conjugated to an oligonucleotide that comprises a target-binding-moiety barcode sequence, said target-binding-moiety barcode sequence differing in sequence from target-binding-moiety barcode sequences contained in oligonucleotide components of conjugates that comprise different target binding moieties, wherein the each oligonucleotide component of the plurality of binding moiety-oligonucleotide conjugates is coated with a nucleic acid binding protein;
(b) distributing subpopulations of cells of the population into compartments;
(c) incorporating a cellular identification sequence during an amplification step performed on nucleic acids from the each of the subpopulations of cells of (b), wherein the cellular identification sequence for each subpopulation of (b) differs from the cellular identification sequence of other subpopulations of (b) distributed to other compartments;
(d) pooling the subpopulations to obtain a pooled population of cells;
(e) distributing subpopulations of the pooled population of (d) into compartments;
(f) incorporating a cellular identification sequence during an amplification step performed on nucleic acids from each of the subpopulations of (e), wherein the cellular identification sequence for each subpopulation (e) differs from the cellular identification sequence of other subpopulations distributed to other compartments in step (e); and wherein steps (d)-(f) are optionally repeated; and
(g) quantifying the level of barcode sequences for each binding moiety associated with the cell in the amplified product, thereby quantifying the level of each of the cellular proteins bound to the target binding moiety.

28. The method of claim 27, wherein the nucleic acid binding moiety is a sequence non-specific nucleic acid binding moiety; optionally wherein the nucleic acid binding protein preferentially binds to single stranded DNA.

29. (canceled)

30. The method of claim 27, wherein the nucleic acid binding protein is Escherichia coli SSB or T4 gene 32 protein.

31. The method of claim 27, wherein the plurality of cells of (a) are permeabilized cells; and/or
the method further comprises quantifying the levels of RNA transcripts in the single cell; and/or
the method further comprises massively parallel sequencing for analysis of transposase-accessible chromatin in the single cell, HiC analysis, whole genome sequencing, mitochondrial DNA sequencing, methylation profiling, haplotype analysis, and CRISPR sgRNA sequencing.

32-33. (canceled)

34. The method of claim 27, wherein one or more of the plurality of binding moiety-oligonucleotide conjugates targets a protein on surface of the cell; and/or one or more of the plurality of binding moiety-oligonucleotide conjugates target a nuclear protein.

35. (canceled)

36. A method of quantifying the level of a target molecule in a single cell, the method comprising incubating the single cell with an oligonucleotide conjugate comprising a binding moiety that specifically binds the target molecule conjugated to an oligonucleotide comprising a barcode sequence that identifies the target molecule, wherein the oligonucleotide is coated with a nucleic acid binding protein; and quantifying the level of binding moiety bound to target molecule; or
a method of quantifying a target nucleic acid molecule in a cell, the method comprising hybridizing an oligonucleotide specific for the target nucleic acid to the target nucleic acid molecule, wherein the oligonucleotide is coated with a nucleic acid binding protein; and quantifying the amount of oligonucleotide hybridized to the target molecule.

37-38. (canceled)

39. The method of claim 36, wherein-the nucleic acid binding protein is Escherichia coli SSB or T4 gp32; and/or the target molecule is a protein.

40-46. (canceled)

47. The method of claim 36, wherein the oligonucleotide comprises a detectable label or comprises a region that specifically hybridizes to a complementary oligonucleotide that comprises a detectable label.

48-57. (canceled)

58. An oligonucleotide conjugated to a target-binding moiety that specifically binds to a target cellular molecule, or an oligonucleotide that specifically hybridizes to target nucleic acid, wherein the oligonucleotide is coated with a nucleic acid binding protein.

59. The oligonucleotide of claim 58, wherein the nucleic acid binding protein is Escherichia coli SSB or T4 gp32; and/or the target molecule is a protein and/or the binding moiety is an antibody.

60-63. (canceled)

64. A kit comprising:
(a) (i) a plurality of binding moiety-oligonucleotide conjugates, wherein each conjugate comprises a target binding moiety that specifically binds to a cellular protein, wherein the oligonucleotide comprises an identifier sequence specific to the target binding moiety, and the identifier sequence differs in sequence from the identifier sequences conjugated to binding moieties that specifically bind to other cellular proteins; and (ii) at least one nucleic acid binding protein; or
(b) a plurality of binding moiety-oligonucleotide conjugates, wherein each conjugate comprises a target binding moiety that specifically binds to an intracellular protein, wherein the oligonucleotide comprises a barcode sequence that identifies the target binding moiety, and the identifier sequence differs in sequence from the identifier sequences conjugated to target binding moieties that specifically bind to other intracellular proteins, wherein the oligonucleotide component is coated with a nucleic acid binding protein; optionally
wherein the kit comprises a buffer for binding of the at least one sequence non-specific nucleic acid binding protein to the oligonucleotide and/or a permeabilization buffer and/or primers to amplify the oligo and/or sequencing adapters.

65-66. (canceled)

67. The kit of claim 64, wherein the at least one sequence non-specific nucleic acid binding protein or the nucleic acid binding protein is Escherichia coli SSB or T4 gp32.

68-69. (canceled)
